SEQUENCE LISTING (42 sequences)

SEQ ID NO: 1 - DNA, 1773 bp, Porcine circovirus
ggtacctccgtggattgttctccagcagtcttccaaaattgcaaagtagtaatcctccgatagagagcttctacagctgggacagcagttgaggagtaccattcctggggggcctgattgctggtaatcaaaatactgcgggccaaaaaaggaacagtaccccctttagtctctacagtcaatggataccggtcacacagtctcagtagatcatcccaaggtaaccagccataaaaatcatccaaaacaacaacttcttctccatgatatccatcccaccacttatttctactaggcttccagtaggtgtccctaggctcagcaaaattacgggcccactggctcttcccacaaccgggcgggcccactatgacgtgtacagctgtcttccaatcacgctgctgcatcttcccgctcactttcaaaagttcagccagcccgcggaaatttctcacatacgttacaggaaactgctcggctacagtcaccaaagaccccgtctccaaaagggtactcacagcagtagacaggtcgctgcgcttcccctggttccgcggagctccacactcgataagtatgtggccttctttactgcagtattctttattctgctggtcggttcctttcgctttctcgatgtggcagcgggcaccaaaataccacttcaccttgttaaaagtctgcttcttagcaaaattcgcaaacccctggaggtgaggagttctaccctcttccaaaccttcctcgccacaaacaaaataatcaaaaagggagattggaagctcccgtattttgtttttctcctcctcggaaggattattaagggtgaacacccacctcttatggggttgcgggccgcttttcttgcttggcattttcactgacgctgccgaggtgctgccgctgccgaagtgcgctggtaatactacagcagcgcacttctttcacttttataggatgacgtatccaaggaggcgttaccgcagaagaagacaccgcccccgcagccatcttggccagatcctccgccgccgcccctggctcgtccacccccgccaccgctaccgttggagaaggaaaaatggcatcttcaacacccgcctctcccgcaccttcggatatactgtcaaggctaccacagtcagaacgccctcctgggcggtggacatgatgagatttaatattgacgactttgttcccccgggaggggggaccaacaaaatctctataccctttgaatactacagaataagaaaggttaaggttgaattctggccctgctcccccatcacccagggtgataggggagtgggctccactgctgttattctagatgataactttgtaacaaaggccacagccctaacctatgacccatatgtaaactactcctcccgccatacaatcccccaacccttctcctaccactcccgttacttcacacccaaacctgttcttgactccaccattgattacttccaaccaaataacaaaaggaatcagctttggatgaggctacaaacctctagaaatgtggaccacgtaggcctcggcactgcgttcgaaaacagtatatacgaccaggactacaatatccgtgtaaccatgtatgtacaattcagagaatttaatcttaaagaccccccacttaaaccctaaatgaataaaaataaaaaccattacgatgtgataacaaaaaagactcagtaatttattttatatgggaaaagggcacagggtgggtccactgcttcaaatcggccttcgggtacc

SEQ ID NO: 2 - DNA, 1768 bp, Porcine circovirus
aaatttctgacaaacgttacagggtgctgctctgcaacggtcaccagactcccgctctccaacaaggtactcacagcagtagacaggtcactccgttgtccttgagatcgaggagctccacattcaataagtaagttgccttctttactgcaatattctttattctgctgatcagttcctttggctttctcgatatggcagcgggcacccaaataccacttcactttattaaaagtttgcttcttcacaaaattagcgaacccctggaggtgaggtgttcgtccttcctcattaccctcctcgccaacaataaaataatcaaatagggagattgggagctcccgtattttcttgcgctcgtcttcggaaggattattcagcgtgaacacccaccttttatgtggttggggtccgcttcttccattcttcttgctgggcatgttgctgctgaggtgctgccgaggtgctgccgctgccgaagtgcgctggtaatacttacagcgcacttctttcgttttcagctatgacgtatccaaggaggcgttaccgcagaagaagacaccgcccccgcagccatcttggccagatcctccgccgccgcccctggctcgtccacccccgccaccgctaccgttggagaaggaaaaatggcatcttcaacacccgcctctcccgcaccttcggatatactgtcaaggctaccacagtcagaacgccctcctgggcggtggacatgatgagatttaatattgacgactttgttcccccgggaggggggaccaacaaaatctctataccctttgaatactacagaataagaaaggttaaggttgaattctggccctgctcccccatcacccagggtgataggggagtgggctccactgctgttattctagatgataactttgtaacaaaggccacagccctaacctatgacccatatgtaaactactcctcccgccatacaatcccccaacccttctcctaccactcccgttacttcacacccaaacctgttcttgactccaccattgattacttccaaccaaataacaaaaggaatcagctttggatgaggctacaaacctctagaaatgtggaccacgtaggcctcggcactgcgttcgaaaacagtatatacgaccaggactacaatatccgtgtaaccatgtatgtacaattcagagaatttaatcttaaagaccccccacttaaaccctaaatgaataataaaaaccattacgaagtgataaaaaagactcagtaatttatttcatatggaaattcagggcatgggggggaaagggtgacgaactggcccccttcctccgtggattgttctgtagcattcttccaaaataccaagaaagtaatcctccgatagagagcttctacagctgggacagcagttgaggagtaccattccaacggggtctgattgctggtaatcagaatactgcgggccaaaaaaggtacagttccacctttagtctctacagtcaatggatatcgatcacacagtctcagtagatcatcccacggcagccagccataaaagtcatcaataacaaccacttcttcaccatggtaaccatcccaccacttgtttctaggtggtttccagtatgtggtttccgggtctgcaaaattagcagcccatttgcttttaccacacccaggtggccccacaatgacgtgtacattggtcttccaatcacgcttctgcattttcccgctcactttcaaaagttcagccagcccgcgg

SEQ ID NO: 3 - DNA, 702 bp, Porcine circovirus
atgacgtatccaaggaggcgttaccgcagaagaagacaccgcccccgcagccatcttggccagatcctccgccgccgcccctggctcgtccacccccgccaccgctaccgttggagaaggaaaaatggcatcttcaacacccgcctctcccgcaccttcggatatactgtcaaggctaccacagtcagaacgccctcctgggcggtggacatgatgagatttaatattgacgactttgttcccccgggaggggggaccaacaaaatctctataccctttgaatactacagaataagaaaggttaaggttgaattctggccctgctcccccatcacccagggtgataggggagtgggctccactgctgttattctagatgataactttgtaacaaaggccacagccctaacctatgacccatatgtaaactactcctcccgccatacaatcccccaacccttctcctaccactcccgttacttcacacccaaacctgttcttgactccaccattgattacttccaaccaaataacaaaaggaatcagctttggatgaggctacaaacctctagaaatgtggaccacgtaggcctcggcactgcgttcgaaaacagtatatacgaccaggactacaatatccgtgtaaccatgtatgtacaattcagagaatttaatcttaaagaccccccacttaaaccctaa

SEQ ID NO: 4 - protein, 233 aa, Porcine circovirus
MTYPRRRYRRRRHRPRSHLGQILRRRPWLVHPRHRYRWRRKNGIFNTRLSRTFGYTVKATTVRTPSWAVDMMRFNIDDFVPPGGGTNLISIPFEYYRIRKVKVEFWPCSPITQGDRGVGSTAVILDDNFVTKATALTYDPYVNYSSRHTIPQPFSYHSRYFTPKPVLDSTIDYFQPNNKRNQLWMRLQTSRNVDHVGLGTAFENSIYDQDYNIRVTMYVQFREFNLKDPPLKP

SEQ ID NO: 5 - DNA, 1768 bp, Porcine circovirus
aattcaaccttaaccttttttattctgtagtattcaaagggtatagagattttgttggtcccccctcccgggggaacaaagtcgtcaatattaaatctcatcatgtccaccgcccaggagggcgttctgactgtggtagccttgacagtatatccgaaggtgcgggagaggcgggtgttgaagatgccatttttccttctccaacggtagcggtggcgggggtggacgagccaggggcggcggcggaggatctggccaagatggctgcgggggcggtgtcttcttctgcggtaacgcctccttggatacgtcatagctgaaaacgaaagaagtgcgctgtaagtattaccagcgcacttcggcagcggcagcacctcggcagcacctcagcagcaacatgcccagcaagaagaatggaagaagcggaccccaaccacataaaaggtgggtgttcacgctgaataatccttccgaagacgagcgcaagaaaatacgggagctcccaatctccctatttgattattttattgttggcgaggagggtaatgaggaaggacgaacacctcacctccaggggttcgctaattttgtgaagaagcaaacttttaataaagtgaagtggtatttgggtgcccgctgccacatcgagaaagccaaaggaactgatcagcagaataaagaatattgcagtaaagaaggcaacttacttattgaatgtggagctcctcgatctcaaggacaacggagtgacctgtctactgctgtgagtaccttgttggagagcgggagtctggtgaccgttgcagagcagcaccctgtaacgtttgtcagaaatttccgcgggctggctgaacttttgaaagtgagcgggaaaatgcagaagcgtgattggaagaccaatgtacacgtcattgtggggccacctgggtgtggtaaaagcaaatgggctgctaattttgcagacccggaaaccacatactggaaaccacctagaaacaagtggtgggatggttaccatggtgaagaagtggttgttattgatgacttttatggctggctgccgtgggatgatctactgagactgtgtgatcgatatccattgactgtagagactaaaggtggaactgtaccttttttggcccgcagtattctgattaccagcaatcagaccccgttggaatggtactcctcaactgctgtcccagctgtagaagctctctatcggaggattacttccttggtattttggaagaatgctacagaacaatccacggaggaagggggccagttcgtcaccctttcccccccatgccctgaatttccatatgaaataaattactgagtcttttttatcacttcgtaatggtttttattattcatttagggtttaagtggggggtctttaagattaaattctctgaattgtacatacatggttacacggatattgtagtcctggtcgtatatactgttttcgaacgcagtgccgaggcctacgtggtccacatttctagaggtttgtagcctcagccaaagctgattccttttgttatttggttggaagtaatcaatagtggagtcaagaacaggtttgggtgtgaagtaacgggagtggtaggagaagggttgggggattgtatggcgggaggagtagtttacatatgggtcataggttagggctgtggcctttgttacaaagttatcatctagaataacagcagtggagcccactcccctatcaccctgggtgatgggggagcagggccag

SEQ ID NO: 6 - protein, 233 aa, Porcine circovirus
MTYPRRRYRRRRHRPRSHLGQILRRRPWLVHPRHRYRWRRKNGIFNTRLSRTFGYTVKATTVRTPSWAVDMMRFNIDDFVPPGGGTNKISIPFEYYRIKKVKVEFWPCSPITQGDRGVGSTAVILDDNFVTKATALTYDPYVNYSSRHTIPQPFSYHSRYFTPKPVLDSTIDYFQPNNKRNQLWLRLQTSRNVDHVGLGTAFENSIYDQDYNIRVTMYVQFREFNLKDPPLKP

SEQ ID NO: 7 - DNA, 1768 bp, Porcine circovirus
aattcaaccttaacctttcttattctgtagtattcaaagggtatagagattttgttggtcccccctcccgggggaacaaagtcgtcaattttaaatctcatcatgtccaccgcccaggagggcgttgtgactgtggtacgcttgacagtatatccgaaggtgcgggagaggcgggtgttgaagatgccatttttccttctccaacggtagcggtggcgggggtggacgagccaggggcggcggcggaggatctggccaagatggctgcgggggcggtgtcttcttctgcggtaacgcctccttggatacgtcatagctgaaaacgaaagaagtgcgctgtaagtattaccagcgcacttcggcagcggcagcacctcggcagcacctcagcagcaacatgcccagcaagaagaatggaagaagcggaccccaaccacataaaaggtgggtgttcacgctgaataatccttccgaagacgagcgcaagaaaatacgggagctcccaatctccctatttgattattttattgttggcgaggagggtaatgaggaaggacgaacacctcacctccaggggttcgctaattttgtgaagaagcaaacttttaataaagtgaagtggtatttgggtgcccgctgccacatcgagaaagccaaaggaactgatcagcagaataaagaatattgcagtaaagaaggcaacttacttattgaatgtggagctcctcgatctcaaggacaacggagtgacctgtctactgctgtgagtaccttgttggagagcgggagtctggtgaccgttgcagagcagcaccctgtaacgtttgtcagaaatttccgcgggctggctgaacttttgaaagtgagcgggaaaatgcagaagcgtgattggaagaccaatgtacacgtcattgtggggccacctgggtgtggtaaaagcaaatgggctgctaattttgcagacccggaaaccacatactggaaaccacctagaaacaagtggtgggatggttaccatggtgaagaagtggttgttattgatgacttttatggctggctgccgtgggatgatctactgagactgtgtgatcgatatccattgactgtagagactaaaggtggaactgtaccttttttggcccgcagtattctgattaccagcaatcagaccccgttggaatggtactcctcaactgctgtcccagctgtagaagctctctatcggaggattacttccttggtattttggaagaatgctacagaacaatccacggaggaagggggccagttcgtcaccctttcccccccatgccctgaatttccatatgaaataaattactgagtcttttttatcacttcgtaatggtttttattattcatttagggtttaagtggggggtctttaagattaaattctctgaattgtacatacatggttacacggatattgtagtcctggtcgtatttactgttttcgaacgcagcgccgaggcctacgtggtccacatttccagaggtttgtagtctcagccaaagctgattccttttgttatttggttggaagtaatcaatagtggagtcaagaacaggtttgggtgtgaagtaacgggagtggtaggagaagggttgggggattgtatggcgggaggagtagtttacatatgggtcataggttagggctgtggcctttgttacaaagttatcatctagaataacagcagtggagcccactcccctatcaccctgggtgatgggggagcagggccag

SEQ ID NO: 8 - protein, 233 aa, Porcine circovirus
MTYPRRRYRRRRHRPRSHLGQILRRRPWLVHPRHRYRWRRKNGIFNTRLSRTFGYTVKRTTVTTPSWAVDMMRFKIDDFVPPGGGTNKISIPFEYYRIRKVKVEFWPCSPITQGDRGVGSTAVILDDNFVTKATALTYDPYVNYSSRHTIPQPFSYHSRYFTPKPVLDSTIDYFQPNNKRNQLWLRLQTSGNVDHVGLGAAFENSKYDQDYNIRVTMYVQFREFNLKDPPLKP

SEQ ID NO: 9 - DNA, 1768 bp, Porcine circovirus
cttttttatcacttcgtaatggtttttattattcatttagggtttaagtggggggtctttaagattaaattctctgaattgtacatacatggttacacggatattgtagtcctggtcgtatatactgttttcgaacgcagtgccgaggcctacgtggtccacatttctagaggtttgtagcctcatccaaagctgattccttttgttatttggttggaagtaatcaatggtggagtcaagaacaggtttgggtgtgaagtaacgggagtggtaggagaagggttgggggattgtatggcgggaggagtagtttacatatgggtcataggttagggctgtggcctttgttacaaagttatcatctagaataacagcagtggagcccactcccctatcaccctgggtgatgggggagcagggccagaattcaaccttaacctttcttattctgtagtattcaaagggtatagagattttgttggtcccccctcccgggggaacaaagtcgtcaatattaaatctcatcatgtccaccgcccaggagggcgttctgactgtggtagccttgacagtatatccgaaggtgcgggagaggcgggtgttgaagatgccatttttccttctccaacggtagcggtggcgggggtggacgagccaggggcggcggcggaggatctggccaagatggctgcgggggcggtgtcttcttctgcggtaacgcctccttggatacgtcatagctgaaaacgaaagaagtgcgctgtaagtattaccagcgcacttcggcagcggcagcacctcggcagcacctcagcagcaacatgcccagcaagaagaatggaagaagcggaccccaaccacataaaaggtgggtgttcacgctgaataatccttccgaagacgagcgcaagaaaatacgggagctcccaatctccctatttgattattttattgttggcgaggagggtaatgaggaaggacgaacacctcacctccaggggttcgctaattttgtgaagaagcaaacttttaataaagtgaagtggtatttgggtgcccgctgccatatcgagaaagccaaaggaactgatcagcagaataaagaatattgcagtaaagaaggcaacttacttattgaatgtggagctcctcgatctcaaggacaacggagtgacctgtctactgctgtgagtaccttgttggagagcgggagtctggtgaccgttgcagagcagcaccctgtaacgtttgtcagaaatttccgcgggctggctgaacttttgaaagtgagcgggaaaatgcagaagcgtgattggaagaccaatgtacacgtcattgtggggccacctgggtgtggtaaaagcaaatgggctgctaattttgcagacccggaaaccacatactggaaaccacctagaaacaagtggtgggatggttaccatggtgaagaagtggttgttattgatgacttttatggctggctgccgtgggatgatctactgagactgtgtgatcgatatccattgactgtagagactaaaggtggaactgtaccttttttggcccgcagtattctgattaccagcaatcagaccccgttggaatggtactcctcaactgctgtcccagctgtagaagctctctatcggaggattactttcttggtattttggaagaatgctacagaacaatccacggaggaagggggccagttcgtcaccctttcccccccatgccctgaatttccatatgaaataaattactgagt

SEQ ID NO: 10 - protein, 233 aa, Porcine circovirus
MTYPRRRYRRRRHRPRSHLGQILRRRPWLVHPRHRYRWRRKNGIFNTRLSRTFGYTVKATTVRTPSWAVDMMRFNIDDFVPPGGGTNKISIPFEYYRIRKVKVEFWPCSPITQGDRGVGSTAVILDDNFVTKATALTYDPYVNYSSRHTIPQPFSYHSRYFTPKPVLDSTIDYFQPNNKRNQLWMRLQTSRNVDHVGLGTAFENSIYDQDYNIRVTMYVQFREFNLKDPPLKP

SEQ ID NO: 11 - DNA, 1767 bp, Porcine circovirus
cttttttatcacttcgtaatggtttttattattcattaagggttaagtggggggtctttaaaattaaattctctgaattgtacatacatggttacacggatattgtattcctggtcgtatatactgttttcgaacgcagtgccgaggcctacgtggtctacatttccagcagtttgtagtctcagccacagctggtttcttttgttgtttggttggaagtaatcaatagtggaatctaggacaggtttgggggtaaagtaccgggagtggtaggagaagggctgggttatggtatggcgggaggagtagtttacataggggtcataggtgagggctgtggcctttgttacaaagttatcatctagaataacagcactggagcccactcccctgtcaccctgggtgatcggggagcagggccagaattcaaccttaacctttcttattctgtagtattcaaagggcacagagcgggggtttgagccccctcctgggggaagaaagtcattaatattgaatctcatcatgtccaccgcccaggagggcgttctgactgtggttcgcttgacagtatatccgaaggtgcgggagaggcgggtgttgaagatgccatttttccttctccagcggtaacggtggcgggggtggacgagccaggggcggcggcggaggatctggccaagatggctgcgggggcggtgtcttcttcttcggtaacgcctccttggatacgtcatatctgaaaacgaaagaagtgcgctgtaagtattaccagcgcacttcggcagcggcagcacctcggcagcacctcagcagcaacatgcccagcaagaagaatggaagaagcggaccccaaccccataaaaggtgggtgttcactctgaataatccttccgaagacgagcgcaagaaaatacgggatcttccaatatccctatttgattattttattgttggcgaggagggtaatgaggaaggacgaacacctcacctccaggggttcgctaattttgtgaagaagcagacttttaataaagtgaagtggtatttgggtgcccgctgccacatcgagaaagcgaaaggaacagatcagcagaataaagaatactgcagtaaagaaggcaacttactgatggagtgtggagctcctagatctcagggacaacggagtgacctgtctactgctgtgagtaccttgttggagagcgggagtctggtgaccgttgcagagcagcaccctgtaacgtttgtcagaaatttccgcgggctggctgaacttttgaaagtgagcgggaaaatgcagaagcgtgattggaagactaatgtacacgtcattgtggggccacctgggtgtggtaaaagcaaatgggctgctaattttgcagacccggaaaccacatactggaaaccacctagaaacaagtggtgggatggttaccatggtgaagaagtggttgttattgatgacttttatggctggctgccctgggatgatctactgagactgtgtgatcgatatccattgactgtagagactaaaggtggaactgtaccttttttggcccgcagtattctgattaccagcaatcagaccccgttggaatggtactcctcaactgctgtcccagctgtagaagctctttatcggaggattacttccttggtattttggaagaatgctacagaacaatccacggaggaagggggccagttcgtcaccctttcccccccatgccctgaatttccatatgaaataaattactgagt

SEQ ID NO: 12 - protein, 233 aa, Porcine circovirus
MTYPRRRYRRRRHRPRSHLGQILRRRPWLVHPRHRYRWRRKNGIFNTRLSRTFGYTVKRTTVRTPSWAVDMMRFNINDFLPPGGGSNPRSVPFEYYRIRKVKVEFWPCSPITQGDRGVGSSAVILDDNFVTKATALTYDPYVNYSSRHTITQPFSYHSRYFTPKPVLDSTIDYFQPNNKRNQLWLRLQTAGNVDHVGLGTAFENSIYDQEYNIRVTMYVQFREFNFKDPPLNP

SEQ ID NO: 13 - DNA, 1767 bp, Porcine circovirus
accagcgcacttcggcagcggcagcacctcggcagcacttcagcagcaacatgcccagcaagaagaatggaagaagcggaccccaaccccataaaaggtgggtgttcactctgaataatccttccgaagacgagcgcaagaaaatacgggatcttccaatatccctatttgattattttattgttggcgaggagggtaatgaggaaggacgaacacctcacctccaggggttcgctaattttgtgaagaagcagacttttaataaggtgaagtggtatttgggtgcccgctgccacatcgagaaagccaaaggaacagatcagcagaataaagaatactgcagtaaagaaggcaacttactgattgagtgtggagctcctagatctcagggacaacggagtgacctgtctactgctgtgagtaccttgttggagagcgggagtctggtgaccgttgcagagcagtaccctgtaacgtttgtcagaaatttccgcgggctggctgaacttttgaaagtgagcgggaaaatgcagaagcgtgattggaagactaatgtacacgtcattgtggggccacctgggtgtggtaaaagcaaatgggctgctaattttgcagacccggaaaccacatactggaaaccacctagaaacaagtggtgggatggttaccatggtgaagaagtggttgttattgatgacttttatggctggctgccctgggatgatctactgagactgtgtgatcgatatccattgactgtagagactaaaggtggaactgtaccttttttggcccgcagtattctgattaccagcaatcagaccccgttggaatggtactcctcaactgctgtcccagctgtagaagctctttatcggaggattacttccttggtattttggaagaatgctacagaacaatccacggaggaagggggccagttcgtcaccctttcccccccatgccctgaatttccatatgaaataaattactgagtcttttttatcacttcgtaatggtttttattattcattaagggttaagtggggggtctttaagattaaattctctgaattgtacatacatggttacacggatattgtattcctggtcgtatatactgttttcgaatgcagtgccgaggcctacgtggtctacatttccagcagtttgtagtctcagccacagctggtttcttttgttgtttggttggaagtaatcaatagtggaatctaggacaggtttgggggtaaagtagcgggagtggtaggagaagggctgggttatggtatggcgggaggagtagtttacataggggtcataggtgagggctgtggcctttgttacaaagttatcatctagaataacagcactggagcccactcccctgtcaccctgggtgatcggggagcagggccagaattcaaccttaacctttcttattctgtagtattcaaagggcacagagcgggggtttgagccccctcctgggggaagaaagtcattaatattgaatctcatcatgtccaccgcccaggagggcgttctgactgtggttcgcttgatagtatatccgaaggtgcgggataggcgggtgttgaagatgccatttttccttctccagcggtaacggtggcgggggtggacgagccaggggcggcggcggaggatctggccaagatggctgcgggggcggtgtcttcttctccggtaacgcctccttggatacgtcatatctgaaaacgaaagaagtgcgctgtaagtatt

SEQ ID NO: 14 - protein, 233 aa, Porcine circovirus
MTYPRRRYRRRRHRPRSHLGQILRRRPWLVHPRHRYRWRRKNGIFNTRLSRTFGYTIKRTTVRTPSWAVDMMRFNINDFLPPGGGSNPRSVPFEYYRIRKVKVEFWPCSPITQGDRGVGSSAVILDDNFVTKATALTYDPYVNYSSRHTITQPFSYHSRYFTPKPVLDSTIDYFQPNNKRNQLWLRLQTAGNVDHVGLGTAFENSIYDQEYNIRVTMYVQFREFNLKDPPLNP

SEQ ID NO: 15 - DNA, 1767 bp, Porcine circovirus
accagcgcacttcggcagcggcagcacctcggcagcacttcagcagcaacatgcccagcaagaagaatggaagaagcggaccccaaccccataaaaggtgggtgttcactctgaataatccttccgaagacgagcgcaagaaaatacgggatcttccaatatccctatttgattattttattgttggcgaggagggtaatgaggaaggacgaacacctcacctccaggggttcgctaattttgtgaagaagcagacttttaataaagtgaagtggtatttgggtgcccgctgccacatcgagaaagccaaaggaacagatcagcagaataaagaatactgcagtaaagaaggcaacttactgattgagtgtggagctcctagatctcagggacaacggagtgacctgtctactgctgtgagtaccttgttggagagcgggagtctggtgaccgttgcagagcagtaccctgtaacgtttgtcagaaatttccgcgggctggctgaacttttgaaagtgagcgggaaaatgcagaagcgtgattggaagactaatgtacacgtcattgtggggccacctgggtgtggtaaaagcaaatgggctgctaattttgcagacccggaaaccacatactggaaaccacctagaaacaagtggtgggatggttaccatggtgaagaagtggttgttattgatgacttttatggctggctgccctgggatgatctactgagactgtgtgatcgatatccattgactgtagagactaaaggtggaactgtaccttttttggcccgcagtattctgattaccagcaatcagaccccgttggaatggtactcctcaactgctgtcccagctgtagaagctctttatcggaggattacttccttggtattttggaagaatgctacagaacaatccacggaggaagggggccagttcgtcaccctttcccccccatgccctgaatttccatatgaaataaattactgagtcttttttatcacttcgtaatggtttttattattcattaagggttaagtggggggtctttaagattaaattctctgaattgtacatacatggttacacggatattgtattcctggtcgtatatactgttttcgaatgcagtgccgaggcctacgtggtctacatttccagcagtttgtagtctcagccacagctggtttcttttgttgtttggttggaagtaatcaatagtggaatctaggacaggtttgggggtaaagtagcgggagtggtaggagaagggctgggttatggtatggcgggaggagtagtttacataggggtcataggtgagggctgtggcctttgttacaaagttatcatctagaataacagcactggagcccactcccctgtcaccctgggtgatcggggagcagggccagaattcaaccttaacctttcttattctgtagtattcaaagggcacagagcgggggtttgagccccctcctgggggaagaaagtcattaatattgaatctcatcatgtccaccgcccaggagggcgttctgactgtggttcgcttgatagtatatccgaaggtgcgggataggcgggtgttgaagatgccatttttccttctccagcggtaacggtggcgggggtggacgagccaggggcggcggcggaggatctggccaagatggctgcgggggcggtgtcttcttctccggtaacgcctccttggatacgtcatatctgaaaacgaaagaagtgcgctgtaagtatt

SEQ ID NO: 16 - protein, 233 aa, Porcine circovirus; identical to SEQ ID NO: 14

SEQ ID NO: 17 - DNA, 1767 bp, Porcine circovirus
accagcgcacttcggcagcggcagcacctcggcagcacttcagcagcaacatgcccagcaagaagaatggaagaagcggaccccaaccccataaaaggtgggtgttcactctgaataatccttccgaagacgagcgcaagaaaatacgggatcttccaatatccctatttgattattttattgttggcgaggagggtaatgaggaaggacgaacacctcacctccaggggttcgctaattttgtgaagaagcagacttttaataaagtgaagtggtatttgggtgcccgctgccacatcgagaaagccaaaggaacagatcagcagaataaagaatactgcagtaaagaaggcaacttactgattgagtgtggagctcctagatctcagggacaacggagtgacctgtctactgctgtgagtaccttgttggagagcgggagtctggtgaccgttgcagagcagtaccctgtaacgtttgtcagaaatttccgcgggctggctgaacttttgaaagtgagcgggaaaatgcagaagcgtgattggaagactaatgtacacgtcattgtggggccacctgggtgtggtaaaagcaaatgggctgctaattttgcagacccggaaaccacatactggaaaccacctagaaacaagtggtgggatggttaccatggtgaagaagtggttgttattgatgacttttatggctggctgccctgggatgatctactgagactgtgtgatcgatatccattgactgtagagactaaaggtggaactgtaccttttttggcccgcagtattctgattaccagcaatcagaccccgttggaatggtactcctcaactgctgtcccagctgtagaagctctttatcggaggattacttccttggtattttggaagaatgctacagaacaatccacggaggaagggggccagttcgtcaccctttcccccccatgccctgaatttccatatgaaataaattactgagtcttttttatcacttcgtaatggtttttattattcattaagggttaagtggggggtctttaagattaaattctctgaattgtacatacatggttacacggatattgtattcctggtcgtatatactgttttcgaacgcagtgccgaggcctacgtggtctacatttccagcagtttgtagtctcagccacagctggtttcttttgttgtttggttggaagtaatcaatagtggaatctaggacaggtttgggggtaaagtagcgggagtggtaggagaagggctgggttatggtatggcgggaggagtagtttacataggggtcataggtgagggctgtggcctttgttacaaagttatcatctagaataacagcactggagcccactcccctgtcaccctgggtgatcggggagcagggccagaattcaaccttaacctttcttattctgtagtattcaaagggcacagagcgggggtttgagccccctcctgggggaagaaagtcattaatattgaatctcatcatgtccaccgcccaggagggcgttctgactgtggttcgcttgatagtatatccgaaggtgcgggataggcgggtgttgaagatgccatttttccttctccagcggtaacggtggcgggggtggacgagccaggggcggcggcggaggatctggccaagatggctgcgggggcggtgtcttcttctccggtaacgcctccttggatacgtcatatctgaaaacgaaagaagtgcgctgtaagtatt

SEQ ID NO: 18 - protein, 233 aa, Porcine circovirus; identical to SEQ ID NO: 14

SEQ ID NO: 19 - DNA, 30 bp, Artificial
  Oligonucleotide primer
gaaccgcgggctggctgaacttttgaaagt

SEQ ID NO: 20 - DNA, 30 bp, Artificial
  Oligonucleotide primer
gcaccgcggaaatttctgacaaacgttaca

SEQ ID NO: 21 - DNA, 21 bp, Artificial
  Oligonucleotide primer
tttggtacccgaaggccgatt

SEQ ID NO: 22 - DNA, 24 bp, Artificial
  Oligonucleotide primer
attggtacctccgtggattgttct

SEQ ID NO: 23 - DNA, 39 bp, Artificial
  Oligonucleotide primer
gaagttaaccctaaatgaataaaaataaaaaccattacg

SEQ ID NO: 24 - DNA, 37 bp, Artificial
  Oligonucleotide primer
ggtggcgcctccttggatacgtcatcctataaaagtg

SEQ ID NO: 25 - DNA, 30 bp, Artificial
  Oligonucleotide primer
aggttataagtggggggtctttaagattaa

SEQ ID NO: 26 - DNA, 27 bp, Artificial
  Oligonucleotide primer
ggaaacgttaccgcagaagaagacacc

SEQ ID NO: 27 - DNA, 35 bp, Artificial
  Oligonucleotide primer
actatagatctttattcatttagagggtctttcag

SEQ ID NO: 28 - DNA, 29 bp, Artificial
  Oligonucleotide primer
tacgggcatgcatgacgtggccaaggagg

SEQ ID NO: 29 - DNA, 36 bp, Artificial
  Oligonucleotide primer
agacgagatctatgaataataaaaaccattacgaag

SEQ ID NO: 30 - DNA, 31 bp, Artificial
  Oligonucleotide primer
cgtaagcatgcagctgaaaacgaaagaagtg

SEQ ID NO: 31 - DNA, 24 bp, Artificial
  Oligonucleotide primer
gctgaacttttgaaagtgagcggg

SEQ ID NO: 32 - DNA, 26 bp, Artificial
  Oligonucleotide primer
tcacacagtctcagtagatcatccca

SEQ ID NO: 33 - DNA, 21 bp, Artificial
  Oligonucleotide primer
ccaactttgtaaccccctcca

SEQ ID NO: 34 - DNA, 18 bp, Artificial
  Oligonucleotide primer
gtggacccaccctgtgcc

SEQ ID NO: 35 - DNA, 20 bp, Artificial
  Oligonucleotide primer
ccagctgtggctccatttaa

SEQ ID NO: 36 - DNA, 29 bp, Artificial
  Oligonucleotide primer
ttcccatataaaataaattactgagtctt

SEQ ID NO: 37 - DNA, 19 bp, Artificial
  Oligonucleotide primer
cagtcagaacgccctcctg

SEQ ID NO: 38 - DNA, 22 bp, Artificial
  Oligonucleotide primer
cctagaaacaagtggtgggatg

SEQ ID NO: 39 - DNA, 19 bp, Artificial
  Oligonucleotide primer
ttgtaacaaaggccacagc

SEQ ID NO: 40 - DNA, 23 bp, Artificial
  Oligonucleotide primer
gtgtgatcgatatccattgactg

SEQ ID NO: 41 - DNA, 20 bp, Artificial
  Oligonucleotide primer
atgcccagcaagaagaatgg

SEQ ID NO: 42 - DNA, 22 bp, Artificial
  Oligonucleotide primer
tggtttccagtatgtggtttcc

(19) United States
(12) Patent Application Publication
(10) Pub. No.: US 2009/0017064 A1
(43) Pub. Date: Jan. 15, 2009

(54) Methods and Compositions for Immunizing Pigs Against Porcine Circovirus

(75) Inventors: Stephen Qitu Wu, Fort Dodge, IA (US); Michael A. Gill, Fort Dodge, IA (US); Hsien-Jue Chu, Bonner Springs, KS (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/170,121

(22) Filed: Jul. 9, 2008

Related U.S. Application Data
(60) Provisional application No. 60/959,131, filed on Jul. 10, 2007.

(51) Int. Cl. A61K 39/12; A61P 31/12

(52) U.S. Cl. 424/205.1; 424/204.1

(57) ABSTRACT

The present invention relates to compositions and methods of eliciting a cross-protective immune response against a pathogenic porcine circovirus by administering to a pig an immunogenically effective amount of a type 1-type 2 chimeric porcine circovirus vaccine. The chimeric vaccine utilized for cross-protection may be administered as a single dose or as multiple doses. The invention further relates to protection of the pig from any one or more of the symptoms or sequelae associated with postweaning multisystemic wasting syndrome (PMWS). Moreover, the administering of the chimeric vaccine also results in reduction in the higher than average mortality associated with the high mortality type 2B strains of porcine circovirus.

### CROSS-REFERENCE TO RELATED APPLICATIONS

This application claims the benefit under 35 U.S.C. §119(e) of U.S. Provisional Application No. 60/959,131 filed Jul. 10, 2007, the disclosure of which is incorporated by reference herein in its entirety.

### FIELD OF THE INVENTION

The present invention relates to the field of animal health and provides methods and compositions for protecting pigs against virulent, high mortality type-2B strains of porcine circovirus. More particularly, the present invention relates to methods for eliciting a cross-protective immune response to a pathogenic porcine circovirus by administering a composition comprising an immunogenically effective amount of a type 2 porcine circovirus vaccine.

### BACKGROUND OF THE INVENTION

Porcine circovirus (PCV) is a small icosahedral non-enveloped virus that contains a single stranded circular DNA genome of about 1.76 kb. It was originally isolated as a cell culture contaminant of a porcine kidney cell line PK-15 (I. Tischer et al., Nature 295:64-66 (1982); I. Tischer et al., Zentralbl. Bakteriol. Hyg. Otg. A. 226(2):153-167 (1974)). PCV is classified in the family of Circoviridae, which consists of three other animal circoviruses (chicken anemia virus (CAV), psittacine beak and feather disease virus (PBFDV) and the recently discovered columbid circovirus (CoCV) from pigeons) and three plant circoviruses (banana bunchy top virus, coconut foliar decay virus and subterranean clover stunt virus) (M. R. Bassami et al., Virology 249:453-459 (1998); J. Mankertz et al., Virus Genes 16:267-276 (1998); A. Mankertz et al., Arch. Virol. 145:2469-2479 (2000); B. M. Meehan et al., J. Gen. Virol. 78:221-227 (1997); B. M. Meehan et al., J. Gen. Virol. 79:2171-2179 (1998); D. Todd et al., Arch. Virol. 117:129-135 (1991)). Members of the three previously recognized animal circoviruses (PCV, CAV, and PBFDV) do not share nucleotide sequence homology or antigenic determinants with each other (M. R. Bassami et al., 1998, supra; D. Todd et al., 1991, supra). Experimental infection of pigs with the PK-15 cells-derived PCV did not produce clinical disease and thus, this virus is not considered to be pathogenic to pigs (G. M. Allan et al., Vet. Microbiol. 44:49-64 (1995); I. Tischer et al., Arch. Virol. 91:271-276 (1986)). This nonpathogenic PCV derived from the contaminated PK-15 cell line was designated as porcine circovirus type 1 or PCV1.

Postweaning multisystemic wasting syndrome (PMWS), first described in 1991 (J. C. Harding and E. G. Clark, 1997, supra), is a complex disease of weaning piglets that is becoming increasingly more widespread. PMWS mainly affects pigs between 5-18 weeks of age. Clinical PMWS signs include progressive weight loss, dyspnea, tachypnea, anemia, diarrhea, and jaundice. Mortality rate may vary from 1% to 2%, and up to 40% in some complicated cases in the U.K. (M. Muirhead, Vet. Rec. 150:456 (2002)). Microscopic lesions characteristic of PMWS include granulomatous interstitial pneumonia, lymphadenopathy, hepatitis, and nephritis (G. M. Allan and J. A. Ellis, J. Vet. Diagn. Invest. 12:3-14 (2000); J. C. Harding and E. G. Clark, 1997, supra).

While PCV1 is ubiquitous in pigs, it is not pathogenic to pigs. The primary causative agent of PMWS is usually a pathogenic strain of PCV designated as porcine circovirus type 2 or PCV2 (G. M. Allan et al., Vet. Rec. 142:467-468 (1998); G. M. Allan et al., J. Vet. Diagn. Invest. 10:3-10 (1998); G. M. Allan et al., Vet. Microbiol. 66:115-23 (1999); G. M. Allan and J. A. Ellis, 2000, supra; J. Ellis et al., 1998, supra; A. L. Hamel et al., 1998, supra; B. M. Meehan et al., 1998, supra; I. Morozov et al., 1998, supra). The complete genomic sequence of the PMWS-associated PCV2 has been determined (M. Fenaux et al., J. Clin. Microbiol. 38:2494-503 (2000); A. L. Hamel et al., 1998, supra; J. Mankertz et al., 1998, supra; B. M. Meehan et al., 1997, supra; B. M. Meehan et al., 1998, supra; I. Morozov et al., 1998, supra).

Sequence analyses reveals that the PMWS-associated PCV2 shares only about 75% nucleotide sequence identity with the nonpathogenic PCV1. The ORF2 gene of both the nonpathogenic PCV1 and the pathogenic PCV2 encodes for the major immunogenic viral capsid protein (P. Nawagitgul et al., Immunol. Clin. Diagn. Lab Immunol. 1:33-40 (2002); P. Nawagitgul et al., J. Gen. Virol. 81:2281-2287 (2000)).

Due to its potential impact on the pig industry, the development of a vaccine against PCV2 has become of major importance. For example, U.S. Pat. No. 6,287,856 (Poet et al.) and WO 99/45956 describe nucleic acids from psittacine beak and feather disease virus (BFDV), a circovirus that infects avian species, and from porcine circovirus (PCV). The patent proposes vaccine compositions comprising naked DNA or mRNA and discloses a nucleic acid vector for the transient expression of PCV in a eukaryotic cell comprising a cis-acting transcription or translation regulatory sequence derived from the human cytomegalovirus immediate or early gene enhancer or promoter functionally linked to a nucleic acid of the sequence.

U.S. Pat. No. 6,217,883 (Allan et al.) and French Patent No. 2,781,159B describe the isolation of five PCV strains from pulmonary or ganglionic samples taken from pigs infected with PMWS in Canada, California and France (Brittany), and their use in combination with at least one porcine parvovirus antigen in vaccine/immunogenic compositions. While the proteins encoded by PCV2 open reading frames (ORF) consisting of ORF1 to ORF13 are broadly described in the patent, there is no exemplification of any specific protein exhibiting immunogenic properties. The patent further describes vectors consisting of DNA plasmids, linear DNA molecules and recombinant viruses that contain and express in vivo a nucleic acid molecule encoding the PCV antigen.

Several other references, for example, U.S. Pat. No. 6,391,314 B1; U.S. Pat. No. 6,368,601 B1; French Patent No. 2,769,321; French Patent No. 2,769,322; WO 01/96377 A2; WO 00/01409; WO 99/18214; WO 00/77216 A2; WO 01/16330 A2; WO 99/29871; etc., describe the administration of PCV1 or PCV2 polypeptides or the nucleic acids encoding the polypeptides of various strains as vaccine compositions.

The citation of any reference herein should not be deemed as an admission that such reference is available as prior art to the instant invention.

### SUMMARY OF THE INVENTION

In its broadest aspect, the present invention is directed to methods of eliciting an immune response against a pathogenic porcine circovirus (PCV) by administering to a pig an immunogenically effective amount of a type 2 porcine circovirus (PCV2) immunogenic composition. In one embodiment, the immunogenic composition is a vaccine composition. The vaccine or immunogenic composition can comprise one or more of the following: 1) a live/attenuated, or modified live chimeric PCV; 2) a killed/inactivated chimeric PCV; 3) a PCV DNA vaccine (e.g. a plasmid vector expressing PCV2 ORF2 or chimeric PCV1-2); 4) an inactivated viral vector (e.g. a baculovirus, adenovirus, or poxvirus, such as raccoonpox virus; or a bacterium, such as E. coli), that expresses PCV2 ORF2; or 5) an ORF2 polypeptide or a nucleic acid encoding an ORF2 polypeptide. In one embodiment, the ORF2 polypeptide or the nucleic acid encoding the ORF2 polypeptide may be from a type 2A or type 2B strain and may induce a cross-protective immune response against any pathogenic type 2A or 2B strain, or a pathogenic non-type 2A or 2B strain, such as, but not limited to, a type 2C or 2D strain. The ORF2 polypeptide may be formulated as known to those skilled in the art as a sub-unit vaccine. Alternatively, the nucleic acid encoding the ORF2 polypeptide may be incorporated into any vector known to those skilled in the art for use in delivery to a host or a host cell for expression of the ORF2 polypeptide. In one embodiment, a vaccine or immunogenic composition wherein the ORF 2 gene is obtained from a type 2A strain of porcine circovirus may cross-protect against infections with a porcine type 2B, type 2C or type 2D strain, or any other variant. In one embodiment, a vaccine or immunogenic composition wherein the ORF 2 gene is obtained from a type 2B porcine circovirus may cross-protect against infections with a porcine type 2A, type 2C or type 2D strain, or any other variant. The administering of such vaccine or immunogenic composition results in protecting the pig against low virulence/low mortality type 2A strains, and also results in cross-protection against high virulence/high mortality type 2B strains of pathogenic porcine circoviruses. The vaccine or immunogenic composition utilized may be administered as a single dose or as multiple doses. The administering results in protection of the pig from any one or more of the symptoms or sequelae associated with postweaning multisystemic wasting syndrome (PMWS). Moreover, the administering of the vaccine or immunogenic composition also results in reduction in the higher than average mortality associated with the high virulence/high mortality type 2B strains of porcine circovirus. The invention provides methods of immunizing a pig against a high virulence/high mortality strain of PCV by administering a vaccine or immunogenic composition comprising a PCV having a PCV1 backbone, further comprising nucleic acids encoding one or more antigens from PCV 2. The invention further provides a method of immunizing a pig against viral infection or postweaning multisystemic wasting syndrome (PMWS) caused by a high virulence strain of a type 2 porcine circovirus (PCV2) comprising administering to the pig an immunogenically effective amount of an immunogenic composition comprising an ORF2 polypeptide from a type 2A porcine circovirus, or a nucleic acid encoding the ORF2 polypeptide from a type 2A porcine circovirus, and a pharmaceutically acceptable carrier, wherein the administering of the composition to a pig induces a cross-protective immune response against a high virulence strain of a type 2 porcine circovirus. In one embodiment, the methods of the invention provide for protection of pigs against infection with a high virulence strain of a type 2 porcine circovirus, which is a type 2B strain. In one embodiment, the methods of the invention provide for immunizing pigs against infection with a high virulence strain of type 2 porcine cirovirus, such as a type 2B porcine circovirus, by administering an immunooenic composition comprising an ORF2 polypeptide from a type 2A porcine circovirus, which comprises the amino acid sequence of any one of SEQ ID NOs: 4, 6, 8 or 10, or an ORF2 polypeptide having at least 90% sequence identity to the amino acid sequence of any one of SEQ ID NOs: 4, 6, 8 or 10.

Accordingly, a first aspect of the invention provides a method of immunizing a pig against viral infection or postweaning multisystemic wasting syndrome (PMWS) caused by a high virulence/high mortality strain of PCV2 comprising administering to the pig an immunogenically effective amount of a vaccine or immunogenic composition comprising:
a) an immunogenically effective amount of a type 1-type 2 chimeric porcine circovirus (PCV1-2) comprising a nucleic acid molecule encoding an infectious, nonpathogenic PCV1 which contains an immunogenic open reading frame (ORF) gene of a pathogenic PCV2 in place of an ORF gene of the PCV1 nucleic acid molecule; or
b) a nucleic acid molecule encoding the type 1-type 2 chimeric porcine circovirus of a).

Another aspect of the invention provides a method for reducing mortality in pigs associated with a high virulence/high mortality strain of a type 2B porcine circovirus comprising administering an immunogenically effective amount of a type 1-type 2 chimeric porcine circovirus vaccine or immunogenic composition, or a nucleic acid molecule encoding the type 1-type 2 chimeric porcine circovirus, as described herein, to a pig.

in one embodiment, the invention provides methods for immunizing or protecting pigs against high virulence/high mortality strains of porcine circovirus by administering a vaccine or immunogenic composition comprising a non-toxic, physiologically acceptable carrier and an immunogenically effective amount of a killed/inactivated type 1-type 2 chimeric porcine circovirus, or a live, attenuated type 1-type 2 chimeric porcine circovirus. In one embodiment, the methods of the invention provide for immunizing or protecting a pig against a porcine circovirus infection by administering the vaccine or immunogenic composition, as described above, which further comprises an adjuvant.

In one embodiment, the invention provides methods for immunizing or protecting pigs against high virulence/high mortality strains of porcine circovirus by administering a vaccine or immunogenic composition comprising a non-toxic, physiologically acceptable carrier and an immunogenically effective amount of a nucleic acid encoding a type 1-type 2 chimeric porcine circovirus. In one embodiment, the methods of the invention provide for immunizing or protecting a pig against a porcine circovirus infection by administering the vaccine or immunogenic composition, as described above, which further comprises an adjuvant.

In one embodiment, the invention provides methods for immunizing or protecting a pig against a high virulence/high mortality type 2B strain of porcine circovirus, by administering a vaccine or immunogenic composition comprising a type 1-type 2 chimeric porcine circovirus, or an infectious nucleic acid encoding the type 1-type 2 chimeric porcine circovirus (SEQ ID NO:1), wherein the administering results in amelioration of one or more symptoms of a porcine circovirus infection.

In one embodiment, the invention provides methods for immunizing or protecting a pig against a high virulence/high mortality type 2B strain of porcine circovirus, by administering an immunogenically effective amount of a vaccine or immunogenic composition, wherein the composition comprises a type 1-type 2 chimeric porcine circovirus, or an infectious nucleic acid encoding the type 1-type 2 chimeric porcine circovirus, and wherein the immunogenic ORF gene of a pathogenic PCV2 that replaces an ORF gene of the PCV1 nucleic acid molecule is ORF-2. In one embodiment, the ORF2 gene is from a pathogenic type 2A strain of porcine circovirus. In one embodiment, the ORF-2 gene comprises the nucleotide sequence as set forth in SEQ ID NO: 3 and the protein encoded by the ORF-2 gene comprises the amino acid sequence as set forth in SEQ ID NO: 4.

In one embodiment, the invention provides methods for immunizing or protecting a pig against a high virulence/high mortality type 2B strain of porcine circovirus, comprising administering a vaccine or immunogenic composition comprising a type 1-type 2 chimeric porcine circovirus, or a nucleic acid encoding a type 1-type 2 chimeric porcine circovirus, wherein the chimeric porcine circovirus comprises the nucleotide sequence as set forth in SEQ ID NO: 1, its complementary strand, or a nucleic acid sequence having at least 95% homology to the nucleotide sequence of SEQ ID NO: 1.

In one embodiment, the invention provides methods for immunizing or protecting a pig against a high virulence/high mortality strain of porcine circovirus by administering a vaccine or immunogenic composition, as described herein, that is administered parenterally. In one embodiment, the vaccine or immunogenic composition is administered subcutaneously, intramuscularly, intranasally, transdermally, intrahepatically, or via the intralymphoid route. In one embodiment, the vaccine or immunogenic composition may be administered as a single dose, or as multiple doses.

In one embodiment, the invention provides methods for inducing a cross-protective immune response that is a humoral or a cell-mediated immune response, or both, by administering to a pig a vaccine or immunogenic composition comprising a type 1-type 2 chimeric porcine circovirus or a nucleic acid encoding a type 1-type 2 chimeric porcine circovirus. In one embodiment, the humoral immune response so induced may result in the generation of antibodies that neutralize a type-2A or a virulent type-2B porcine circovirus. In one embodiment, the cell-mediated immune response so induced may result in generation of T cells that are reactive with cells infected with a virulent type-2B porcine circovirus. In one embodiment, the methods of the invention provide for inducing a cross-protective immune response that is observed for a period of at least four months following administration.

In one embodiment, the invention provides methods for immunizing or protecting a pig against a high virulence/high mortality strain of porcine circovirus by administering a vaccine or immunogenic composition comprising a type 1-type 2 chimeric porcine circovirus or a nucleic acid encoding a type 1-type 2 chimeric porcine circovirus, wherein the high virulence/high mortality strain of porcine circovirus is a type-2B porcine circovirus that shares at least 80% nucleic acid sequence homology with a non-virulent strain of type 2A porcine circovirus.

In one embodiment, the invention provides methods for immunizing or protecting a pig against a high virulence/high mortality strain of porcine circovirus by administering a vaccine or immunogenic composition comprising a type 1-type 2 chimeric porcine circovirus or a nucleic acid encoding a type 1-type 2 chimeric porcine circovirus, wherein the high virulence/high mortality strain of porcine circovirus is a type-2B porcine circovirus that shares at least 95% nucleic acid sequence homology with a non-virulent strain of type 2A porcine circovirus. Exemplary sequences encoding certain of the low virulence/low mortality strains of type 2A porcine circovirus include, but are not limited to those found in GenBank accession numbers AF055391 (SEQ ID NO: 5), AF055392 (SEQ ID NO: 7) and AF264042 (SEQ ID NO: 9). Exemplary sequences encoding certain of the high virulence/high mortality strains of type 2B porcine circovirus include, but are not limited to those found in GenBank accession numbers AJ623306 (SEQ ID NO: 11), DQ220727 (SEQ ID NO: 13), DQ220728 (SEQ ID NO: 15), and DQ220739 (SEQ ID NO: 17).

In one embodiment, the invention provides methods for immunizing or protecting a pig against a high virulence/high mortality strain of porcine circovirus by administering a vaccine or immunogenic composition comprising a type 1-type 2 chimeric porcine circovirus or a nucleic acid encoding a type 1-type 2 chimeric porcine circovirus, wherein the high virulence/high mortality strain of porcine circovirus is a type-2B porcine circovirus that shares at least 97% nucleic acid sequence homology with a non-virulent strain of type 2A porcine circovirus.

In one embodiment, the invention provides methods for immunizing or protecting a pig against a high virulence/high mortality strain of porcine circovirus by administering a vaccine or immunogenic composition comprising a type 1-type 2 chimeric porcine circovirus or a nucleic acid encoding a type 1-type 2 chimeric porcine circovirus, wherein the high virulence/high mortality strain of porcine circovirus is a type-2B porcine circovirus that shares at least 99% nucleic acid sequence homology with a non-virulent strain of type 2A porcine circovirus.

In one embodiment, the invention provides methods for immunizing or protecting a pig against a high virulence/high mortality strain of porcine circovirus by administering a vaccine or immunogenic composition comprising a type 1-type 2 chimeric porcine circovirus or a nucleic acid encoding a type 1-type 2 chimeric porcine circovirus, wherein the high virulence/high mortality strain of porcine circovirus is a type-2B porcine circovirus that shares at least 95% nucleic acid sequence homology with the nucleotide sequence of a non-virulent strain of type-2A porcine circovirus as set forth in GenBank accession numbers AF055391, AF055392 and AF264042 (SEQ ID NOs: 5, 7 and 9, respectively)

In one embodiment, the invention provides methods for immunizing or protecting a pig against a high virulence/high mortality strain of porcine circovirus by administering a vaccine or immunogenic composition comprising a type 1-type 2 chimeric porcine circovirus or a nucleic acid encoding a type 1-type 2 chimeric porcine circovirus, wherein the high virulence/high mortality strain of porcine circovirus is a virulent strain of a type-2B porcine circovirus that contains a capsid protein encoded by the ORF 2 gene that exhibits not less than 90% sequence identity with a capsid protein encoded by the ORF 2 gene of a non-virulent strain of a porcine circovirus, such as those described above in GenBank accession numbers AF055391, AF055392 and AF264042. The amino acid sequences of the capsid proteins of these type 2A low virulence/low mortality strains of porcine circovirus are shown in SEQ ID NOs: 6, 8 and 10, respectively.

In one embodiment, the invention provides methods for immunizing or protecting a pig against a high virulence/high mortality strain of porcine circovirus by administering a vaccine or immunogenic composition comprising a type 1-type 2 chimeric porcine circovirus or a nucleic acid encoding a type 1-type 2 chimeric porcine circovirus, wherein the high virulence/high mortality strain of porcine circovirus is a virulent strain of a type-2B porcine circovirus that contains a capsid protein encoded by the ORF 2 gene that exhibits not less than 90% sequence identity with the amino acid sequence of SEQ ID NO: 4. In one embodiment, the ORF 2 gene in the chimeric porcine circovirus derived from a type 2A strain comprises the nucleic acid sequence of SEQ ID NO: 3 and the protein encoded by the ORF2 gene in the chimeric porcine circovirus comprises the amino acid sequence of SEQ ID NO: 4. In one embodiment, the capsid protein encoded by the ORF 2 gene from a non-virulent strain of porcine circovirus comprises the amino acid sequence of any one of SEQ ID NOs: 6, 8 or 10 and the capsid protein encoded by the ORF 2 gene from a virulent strain of a porcine circovirus comprises the amino acid sequence of any one of SEQ ID NOs: 12, 14, 16 or 18.

In one embodiment, the methods of the invention provide for immunizing or protecting a pig from infection with a high virulence/high mortality strain of type 2B porcine circovirus, comprising administering to a pig a vaccine or immunogenic composition comprising a type 1-type 2 chimeric porcine circovirus, or a nucleic acid encoding a type 1-type 2 chimeric porcine circovirus, wherein said administering results in amelioration of one or more of the following clinical symptoms:

reduction of microscopic lesions in one or more lymphoid or non-lymphoid tissues of pigs exposed to a virulent form of a type-2B porcine circovirus;

reduction of viremia associated with a porcine circovirus infection;

reduction in the level of type-2A or type-2B nucleic acid in one or more tissues.

In one embodiment, the methods of the invention further comprise administering to a pig an immunogenically effective amount of a second different vaccine or immunogenic composition prior to, in conjunction with, or subsequent to, administering the chimeric type-1-type 2 porcine circovirus vaccine or immunogenic compositions as described herein. In one embodiment, the second vaccine or immunogenic composition may be protective against other microorganisms that are known to infect pigs, which may include bacteria, viruses, or protozoans. In one embodiment, the second different vaccine or immunogenic composition is protective against a microorganism selected from the group consisting of porcine reproductive and respiratory syndrome virus (PRRS), porcine parvovirus (PPV), Mycoplasma hyopneumoniae, Haemophilus parasuis, Pasteurella multocida, Streptococcum suis, Actinobacillus pleuropneumoniae, Bordetella bronchiseptica, Salmonella choleraesuis, Erysipelothrix rhusiopathiae, leptospira bacteria, swine influenza virus, Escherichia coli antigen, porcine respiratory coronavirus, rotavirus, a pathogen causative of Aujesky's Disease, and a pathogen causative of Swine Transmissible Gastroenteritis.

In one embodiment, the invention provides methods for immunizing or protecting a pig against a high virulence/high mortality type 2B strain of porcine circovirus by administering a vaccine or immunogenic composition comprising a type 1-type 2 chimeric porcine circovirus, or a nucleic acid encoding a type 1-type 2 chimeric porcine circovirus, wherein the capsid protein encoded by the ORF2 gene of a high virulence/high mortality type 2B strain of porcine circovirus has a conservative or non-conservative amino acid substitution at one or more of the following positions of any one of SEQ ID NOs: 6, 8 or 10: position numbers 57, 59, 63, 75, 77, 80, 86, 88, 89, 91, 99, 121, 151, 190, 191, 200, 206, 210, 232.

In one embodiment, the invention provides methods for immunizing or protecting a pig against a high virulence/high mortality type 2B strain of porcine circovirus by administering a vaccine or immunogenic composition comprising a type 1-type 2 chimeric porcine circovirus or a nucleic acid encoding a type 1-type 2 chimeric porcine circovirus, wherein the capsid protein encoded by the ORF 2 gene of a high virulence/high mortality strain of type-2B porcine circovirus has one or more of the following variations:

the isoleucine at position 91 of any one of SEQ ID NOs: 6, 8 or 10 is replaced with a valine; and/or

the lysine at position 99 of SEQ ID NO: 6 is replaced with an arginine.

These and other aspects of the present invention will be better appreciated by reference to the following drawings and Detailed Description.

### BRIEF DESCRIPTION OF THE DRAWINGS

FIG. 1. Average Antibody Titers Post-Vaccination as Measured by IPMA

FIG. 2. Average Antibody Titers Post-Infection as Measured by IPMA

### DETAILED DESCRIPTION

Before the present methods and treatment methodology are described, it is to be understood that this invention is not limited to particular methods, and experimental conditions described, as such methods and conditions may vary. It is also to be understood that the terminology used herein is for purposes of describing particular embodiments only, and is not intended to be limiting.

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth.

Accordingly, in the present application, there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994).

Although any methods and materials similar or equivalent to those described herein can be used in the practice or testing of the invention, the preferred methods and materials are now described. All publications mentioned herein are incorporated by reference in their entirety.

### DEFINITIONS

The terms used herein have the meanings recognized and known to those of skill in the art, however, for convenience and completeness, particular terms and their meanings are set forth below.

By “antigen” is meant a molecule that contains one or more epitopes capable of stimulating a host's immune system to make a cellular antigen-specific immune response or a humoral antibody response when the antigen is presented in accordance with the present invention. Normally, an epitope will include between about 3-15, generally about 5-15, amino acids. Epitopes of a given protein can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715, all incorporated herein by reference in their entireties. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Furthermore, for purposes of the present invention, an “antigen” refers to a protein that includes modifications, such as deletions, additions and substitutions (generally conservative in nature, but they may be non-conservative), to the native sequence, so long as the protein maintains the ability to elicit an immunological response. These modifications may be deliberate, as through site-directed mutagenesis, or through particular synthetic procedures, or through a genetic engineering approach, or may be accidental, such as through mutations of hosts, which produce the antigens.

In general, the term “chimeric protein” refers to a polypeptide consisting of one or more domains from different proteins or mutations within a single protein giving the characteristics of another protein. In the manner of the present invention, the term “chimeric vaccine” generally refers to a vaccine comprising nucleic acid or amino acid sequences obtained from at least two different strains or serotypes of a microorganism. For example, a “type-1-2 chimeric porcine circovirus vaccine” comprises the nucleic acid from a non-pathogenic type 1 circovirus, wherein the ORF2 gene from the type 1 is deleted and replaced with the ORF2 gene from a pathogenic type 2A strain of porcine circovirus. Accordingly, this genetically engineered chimeric vaccine is naturally attenuated in that viral replication may proceed, but since the backbone of the virus is essentially the type 1 non-pathogenic strain, there is no pathology associated with viral replication. Likewise, since the ORF2 gene, which encodes the viral capsid protein, is from a pathogenic type 2A strain, the immune response that is elicited should be specific for the pathogenic type 2A strain.

The term “circovirus”, as used herein, unless otherwise indicated, refers to any strain of circovirus that falls within the family Circoviridae. For example, in the present invention, the circovirus is a pathogenic porcine circovirus. In particular embodiments, the pathogenic porcine circovirus is a low virulent/low mortality type 2A strain of porcine circovirus or a high virulence/high mortality type 2B strain of porcine circovirus.

“Complementary” is understood in its recognized meaning as identifying a nucleotide in one sequence that hybridizes (anneals) to a nucleotide in another sequence according to the rule A→T, U and C→G (and vice versa) and thus “matches” its partner for purposes of this definition. Enzymatic transcription has measurable and well known error rates (depending on the specific enzyme used), thus within the limits of transcriptional accuracy using the modes described herein, in that a skilled practitioner would understand that fidelity of enzymatic complementary strand synthesis is not absolute and that the amplicon need not be completely matched in every nucleotide to the target or template RNA. Procedures using conditions of high stringency are as follows. Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 μg/ml denatured salmon sperm DNA and 5-20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 min before autoradiography. Other conditions of high stringency that may be used are well known in the art. (see, e.g., Sambrook et al., 1989, Molecular Cloning, A Laboratory Manual, 2d Ed., Cold Spring Harbor Laboratory Press, Cold Spring Harbor, N.Y.; see also, Ausubel et al., eds., in the Current Protocols in Molecular Biology series of laboratory technique manuals, 1987-1997 Current Protocols,© 1994-1997 John Wiley and Sons, Inc.).

It is noted that in this disclosure, terms such as “comprises”, “comprised”, “comprising”, “contains”, “containing” and the like can have the meaning attributed to them in U.S. patent law; eg., they can mean “includes”, “included”, “including” and the like. Terms such as “consisting essentially of” and “consists essentially of” have the meaning attributed to them in U.S. patent law, eg., they allow for the inclusion of additional ingredients or steps that do not detract from the novel or basic characteristics of the invention, ie., they exclude additional unrecited ingredients or steps that detract from novel or basic characteristics of the invention, and they exclude ingredients or steps of the prior art, such as documents in the art that are cited herein or are incorporated by reference herein, especially as it is a goal of this document to define embodiments that are patentable, eg., novel, nonobvious, inventive, over the prior art, eg., over documents cited herein or incorporated by reference herein. And, the terms “consists of” and “consisting of” have the meaning ascribed to them in U.S. patent law; namely, that these terms are closed ended.

A “conservative amino acid substitution” refers to the substitution of one or more of the amino acid residues of a protein with other amino acid residues having similar physical and/or chemical properties. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such alterations will not be expected to affect apparent molecular weight as determined by polyacrylamide gel electrophoresis, or isoelectric point. Particularly preferred substitutions are: Lys for Arg and vice versa such that a positive charge may be maintained; Glu for Asp and vice versa such that a negative charge may be maintained; Ser for Thr such that a free —OH can be maintained; and Gln for Asn such that a free NH₂ can be maintained.

The term “cross-protective immune response” refers to the development of a beneficial humoral response and/or a cell-mediated response that is primarily directed against the particular strain of microorganism used as the antigen in the vaccine composition, but which is also directed against, or cross-reacts with, another different strain of that same microorganism. The cross-protective immune response may be a humoral (antibody) and/or a cell-mediated (T cell) immune response. Conceptually, strong and long-lasting cross-protective immunity could be elicited by vaccines that express multiple antigens that are shared among different pathogenic strains (serotypes). The rationale is that although different strains possess different antigen repertoires, some of the protective antigens may be shared among heterologous serotypes, and expression of these shared antigens may lead to cross-protective immunity. For example, in the present invention, the “type-1-2 chimeric porcine circovirus vaccine”, designated “PSV1-2”, or “PSV1/2”, or “cPSV1-2” or “cPSV1/2”, all of which are used interchangeably, was prepared by utilizing the nucleic acid molecule encoding an infectious, but non-pathogenic, PCV1 strain of porcine circovirus and the ORF2 gene from this non-pathogenic PCV1 strain was replaced with the ORF2 gene from a pathogenic PCV2A strain of porcine circovirus, yet this chimeric vaccine was shown to protect pigs against challenge with a high virulence/high mortality type 2B porcine circovirus. The term “infectious” refers to the fact that the virus can replicate in cells in vitro or in vivo.

“Encoded by” refers to a nucleic acid sequence which codes for a polypeptide sequence, wherein the polypeptide sequence contains an amino acid sequence of at least 3 to 5 amino acids, more preferably at least 8 to 10 amino acids, and even more preferably at least 15 to 20 amino acids, a polypeptide encoded by the nucleic acid sequences. Also encompassed are polypeptide sequences, which are immunologically identifiable with a polypeptide encoded by the sequence. Thus, an antigen “polypeptide,” “protein,” or “amino acid” sequence may have at least 70% similarity, preferably at least about 80% similarity, more preferably about 90-95% similarity, and most preferably about 99% similarity, to a polypeptide or amino acid sequence of an antigen.

A “gene” as used in the context of the present invention is a sequence of nucleotides in a nucleic acid molecule (chromosome, plasmid, etc.) with which a genetic function is associated. A gene is a hereditary unit, for example of an organism, comprising a polynucleotide sequence (e.g., a DNA sequence for mammals) that occupies a specific physical location (a “gene locus” or “genetic locus”) within the genome of an organism. A gene can encode an expressed product, such as a polypeptide or a polynucleotide (e.g., tRNA). Alternatively, a gene may define a genomic location for a particular event/function, such as the binding of proteins and/or nucleic acids (e.g., phage attachment sites), wherein the gene does not encode an expressed product. Typically, a gene includes coding sequences, such as polypeptide encoding sequences, and non-coding sequences, such as promoter sequences, poly-adenlyation sequences, transcriptional regulatory sequences (e.g., enhancer sequences). Many eucaryotic genes have “exons” (coding sequences) interrupted by “introns” (non-coding sequences). In certain cases, a gene may share sequences with another gene(s) (e.g., overlapping genes).

The “gnotobiotic” pigs are germ-free pigs.

Thus, “homology” or “identity” or “similarity” refers to sequence similarity between two peptides or between two nucleic acid molecules. Homology can be determined by comparing a position in each sequence, which may be aligned for purposes of comparison. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences. A degree of identity of amino acid sequences is a function of the number of identical amino acids at positions shared by the amino acid sequences. A degree of homology or similarity of amino acid sequences is a function of the number of amino acids, i.e. structurally related, at positions shared by the amino acid sequences. An “unrelated” or “non-homologous” sequence shares less than 40% identity, though preferably less than 25% identity, with one of the sequences of the present invention. Therefore, a “homolog” of a porcine circovirus or a fragment thereof, should share at least about 75% homology with the porcine circovirus or fragment thereof (preferably about 80% homology, more preferably about 90-95% homology and most preferably about 99% homology).

An “immune response” to a vaccine or immunogenic composition is the development in a subject of a humoral and/or a cell-mediated immune response to molecules present in the antigen or vaccine composition of interest. For purposes of the present invention, a “humoral immune response” is an antibody-mediated immune response and involves the generation of antibodies with affinity for the antigen/vaccine of the invention, while a “cell-mediated immune response” is one mediated by T-lymphocytes and/or other white blood cells. A “cell-mediated immune response” is elicited by the presentation of antigenic epitopes in association with Class I or Class II molecules of the major histocompatibility complex (MHC). This activates antigen-specific CD4+ T helper cells or CD8+ cytotoxic T lymphocyte cells (“CTLs”). CTLs have specificity for peptide antigens that are presented in association with proteins encoded by the major histocompatibility complex (MHC) and expressed on the surfaces of cells. CTLs help induce and promote the intracellular destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cell-mediated immune response” also refers to the production of cytokines, chemokines and other such molecules produced by activated T-cells and/or other white blood cells, including those derived from CD4+ and CD8+ T-cells. The ability of a particular antigen or composition to stimulate a cell-mediated immunological response may be determined by a number of assays, such as by lymphoproliferation (lymphocyte activation) assays, CTL cytotoxic cell assays, by assaying for T-lymphocytes specific for the antigen in a sensitized subject, or by measurement of cytokine production by T cells in response to restimulation with antigen. Such assays are well known in the art. See, e.g., Erickson et al., J. Immunol. (1993) 151:4189-4199; Doe et al., Eur. J. Immunol. (1994) 24:2369-2376.

The term “immunogenic” refers to the ability of an antigen or a vaccine to elicit an immune response, either humoral or cell mediated, or both. An “immunogenically effective amount” as used herein refers to the amount of antigen or vaccine sufficient to elicit an immune response, either a cellular (T cell) or humoral (B cell or antibody) response, or both, as measured by standard assays known to one skilled in the art. The effectiveness of an antigen as an immunogen, can be measured either by proliferation assays, by cytolytic assays, such as chromium release assays to measure the ability of a T cell to lyse its specific target cell, or by measuring the levels of B cell activity by measuring the levels of circulating antibodies specific for the antigen in serum. Furthermore, the level of protection of the immune response may be measured by challenging the immunized host with the antigen that has been injected. For example, if the antigen to which an immune response is desired is a virus or a tumor cell, the level of protection induced by the “immunogenically effective amount” of the antigen is measured by detecting the percent survival or the percent mortality after virus or tumor cell challenge of the animals. In one embodiment, an “immunogenically effective amount” of the vaccine or immunogenic composition refers to a titer of virus particles ranging from about 1 to 7 Log₁₀ virus particles/ml as measured by the FAID₅₀ method (King et al., Journal of Comparative Medicine and Vet. Science, 29:85-89 (1965)) and in U.S. Pat. No. 4,824,785. In one embodiment, an “immunogenically effective amount” of the vaccine or immunogenic compositions is a titer of virus particles ranging from about 2 to 5 Log₁₀ virus particles/ml as measured by the FAID₅₀ method (King et al., Journal of Comparative Medicine and Vet. Science, 29:85-89 (1965)) and in U.S. Pat. No. 4,824,785. In one embodiment, an “immunogenically effective amount” of an infectious DNA vaccine or immunogenic composition may range from about 50 to 5000 μg. In one embodiment, an “immunogenically effective amount” of an infectious DNA vaccine or immunogenic composition may range from about 50 to 1000 μg. In certain embodiments, the term “about” means within 20%, preferably within 10%, and more preferably within 5%.

The term “immunogenic composition” relates to any pharmaceutical composition containing an antigen, eg. a microorganism, which composition can be used to elicit an immune response in a mammal. The immune response can include a T cell response, a B cell response, or both a T cell and B cell response. The composition may serve to sensitize the mammal by the presentation of antigen in association with MHC molecules at the cell surface. In addition, antigen-specific T-lymphocytes or antibodies can be generated to allow for the future protection of an immunized host. An “immunogenic composition” may contain a live, attenuated, or killed/inactivated vaccine comprising a whole microorganism or an immunogenic portion derived therefrom that induces either a cell-mediated (T cell) immune response or an antibody-mediated (B cell) immune response, or both, and may protect the animal from one or more symptoms associated with infection by the microorganism, or may protect the animal from death due to the infection with the microorganism.

An “immunogenic ORF” or “immunogenic ORF” refers to an open reading frame that elicits an immune response, for example, ORF2 encodes an immunogenic capsid protein.

The vaccines and immunogenic compositions of the present invention can further comprise one or more additional “immunomodulators”, which are agents that perturb or alter the immune system, such that either up-regulation or down-regulation of humoral and/or cell-mediated immunity is observed. In one particular embodiment, up-regulation of the humoral and/or cell-mediated arms of the immune system is preferred. Examples of certain immunomodulators include, for example, an adjuvant or cytokine, among others. Non-limiting examples of adjuvants that can be used in the vaccine of the present invention include the RIBI adjuvant system (Ribi Inc., Hamilton, Mont.), alum, mineral gels such as aluminum hydroxide gel, oil-in-water emulsions, water-in-oil emulsions such as, e.g., Freund's complete and incomplete adjuvants, Block copolymer (CytRx, Atlanta Ga.), QS-21 (Cambridge Biotech Inc., Cambridge Mass.), SAF-M (Chiron, Emeryville Calif.), AMPHIGEN® adjuvant, saponin, Quil A or other saponin fraction, monophosphoryl lipid A, and Avridine lipid-amine adjuvant. Non-limiting examples of oil-in-water emulsions useful in the vaccine of the invention include modified SEAM62 and SEAM 1/2 formulations. Modified SEAM62 is an oil-in-water emulsion containing 5% (v/v) squalene (Sigma), 1% (v/v) SPAN® 85 detergent (ICI Surfactants), 0.7% (v/v) TWEEN® 80 detergent (ICI Surfactants), 2.5% (v/v) ethanol, 200 μg/ml Quil A, 100 μg/ml cholesterol, and 0.5% (v/v) lecithin. Modified SEAM 1/2 is an oil-in-water emulsion comprising 5% (v/v) squalene, 1% (v/v) SPAN® 85 detergent, 0.7% (v/v) Tween 80 detergent, 2.5% (v/v) ethanol, 100 μg/ml Quil A, and 50 μg/ml cholesterol. Other “immunomodulators” that can be included in the vaccine include, eg., one or more interleukins, interferons, or other known cytokines. In one embodiment, the adjuvant may be a cyclodextrin derivative or a polyanionic polymer, such as those described in U.S. Pat. Nos. 6,165,995 and 6,610,310, respectively.

The term “infectious” means that the virus replicates or is capable of replicating in pigs, regardless of whether or not the virus causes any diseases. In the present invention, an example of an “infectious” DNA is shown as the PCV2 DNA of SEQ ID NO: 2.

The term “isolated” or “purified” means that the material is removed from its original environment (e.g., the natural environment if it is naturally occurring). For example, an “isolated” or “purified” peptide or protein is substantially free of cellular material or other contaminating proteins from the cell or tissue source from which the protein is derived, or substantially free of chemical precursors or other chemicals when chemically synthesized. The language “substantially free of cellular material” includes preparations of a polypeptide/protein in which the polypeptide/protein is separated from cellular components of the cells from which it is isolated or recombinantly produced. Thus, a polypeptide/protein that is substantially free of cellular material includes preparations of the polypeptide/protein having less than about 30%, 20%, 10%, 5%, 2.5%, or 1%, (by dry weight) of contaminating protein. When the polypeptide/protein is recombinantly produced, it is also preferably substantially free of culture medium, i.e., culture medium represents less than about 20%, 10%, or 5% of the volume of the protein preparation. When polypeptide/protein is produced by chemical synthesis, it is preferably substantially free of chemical precursors or other chemicals, i.e., it is separated from chemical precursors or other chemicals which are involved in the synthesis of the protein. Accordingly, such preparations of the polypeptide/protein have less than about 30%, 20%, 10%, 5% (by dry weight) of chemical precursors or compounds other than polypeptide/protein fragment of interest. An “isolated” or “purified” nucleic acid molecule is one which is separated from other nucleic acid molecules which are present in the natural source of the nucleic acid molecule. Moreover, an “isolated” nucleic acid molecule, such as a cDNA molecule or an RNA molecule, can be substantially free of other cellular material, or culture medium when produced by recombinant techniques, or substantially free of chemical precursors or other chemicals when chemically synthesized.

The terms “killed” or “inactivated” are used interchangeably herein and refer to a significant or complete reduction in the infectivity of the virus(es) utilized for preparation of the vaccine compositions. The killing or inactivation of the viruses may be evaluated according to any procedure known to those skilled in the art, for example, by molecular biology methods (PCR), methods for titration of the viral titre, fluorescence, immunological methods (ELISA, RIA and the like), immunoenzymatic methods allowing the detection of one or more viral polypeptides (Western and the like). A number of different inactivating agents and means have been employed including formalin, azide, freeze-thaw, sonication, heat treatment, sudden pressure drop, detergent (especially non-ionic detergents), lysozyme, phenol, proteolytic enzymes and .beta.-propiolactone.

The term “lymphoid tissue” refers to any tissue that is rich in lymphocytes and accessory cells such as macrophages and reticular cells and supported by a meshwork of connective tissue. The lymphoid tissue includes the bone marrow, thymus, lymph nodes, spleen, tonsils, adenoids, Peyer's Patches and lymphocyte aggregates on mucosal surfaces. “Non-lymphoid” tissue refers to any other tissue that is not rich in lymphocytes and accessory cells as defined herein.

A “non-conservative amino acid substitution” refers to the substitution of one or more of the amino acid residues of a protein with other amino acid residues having dissimilar physical and/or chemical properties, using the characteristics defined above.

As used herein, the phrase “nucleic acid” or “nucleic acid molecule” refers to DNA, RNA, as well as any of the known base analogs of DNA and RNA or chimeras formed therefrom. Thus, a “nucleic acid” or a “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”) in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5N to 3N direction along the nontranscribed strand of DNA (i.e., the strand having a sequence homologous to the mRNA). A “recombinant DNA molecule” is a DNA molecule that has undergone a molecular biological manipulation.

A “nucleotide” refers to a subunit of DNA or RNA consisting of nitrogenous bases (adenine, guanine, cytosine and thymine), a phosphate molecule, and a sugar molecule (deoxyribose in DNA and ribose in RNA).

The term “open reading frame” or “ORF”, or “ORF”, as used herein, refers to the minimal nucleotide sequence required to encode a particular circovirus protein or antigen without an intervening stop codon.

The term “parenteral” refers to a substance taken into the body or administered in a manner other than through the digestive tract, for example, as by intravenous or intramuscular injection.

The term “pathogenic” refers to the ability of any agent of infection, such as a bacterium or a virus, to cause disease. In the manner of the present invention, the term “pathogenic” refers to the ability of a porcine circovirus, in particular, a type 2 porcine circovirus, to cause a disease in pigs referred to as “post-weaning multisystemic wasting syndrome” or “PMWS”. This disease is often characterized by wasting or poor performance in weaned pigs and by moderate to severe lymphoid lesions with lymphoid depletion and histiocytic replacement of follicles in lymphoid tissues. Pigs suffering from PMWS are also known to have respiratory disease, for example, interstitial pneumonia, lymphohistiocytic hepatitis and lymphohistiocytic interstitial nephritis. Other conditions associated with a “pathogenic” type 2 porcine circovirus include sporadic reproductive failure, enteritis, and porcine dermatitis and nephropathy syndrome (PDNS). A “non-pathogenic” microorganism refers to a microorganism that lacks the characteristics noted above for the “pathogenic” strains of porcine circovirus. The “non-pathogenic” porcine circovirus is generally referred to as a type 1 porcine circovirus. The “pathogenic” strains of porcine circovirus are generally referred to as type 2 porcine circoviruses. The “non-pathogenic” porcine circovirus is generally referred to as a type 1 porcine circovirus.

The terms “PCV2 plasmid DNA,” “PCV2 genomic DNA” and “PCV2 molecular DNA” are being used interchangeably to refer to the same cloned nucleotide sequence.

Thus, the term “percent identical” or “percent sequence identity” refers to sequence identity between two amino acid sequences or between two nucleotide sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

The term “pharmaceutically acceptable carrier” means a carrier approved by a regulatory agency of a Federal, a state government, or other regulatory agency, or listed in the U.S. Pharmacopeia or other generally recognized pharmacopeia for use in animals, including humans as well as non-human mammals. The term “carrier” refers to a diluent, adjuvant, excipient, or vehicle with which the pharmaceutical composition is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. The formulation should suit the mode of administration.

A “polynucleotide” is a nucleic acid polymer, which typically encodes a biologically active (e.g., immunogenic) protein or polypeptide. Depending on the nature of the polypeptide encoded by the polynucleotide, a polynucleotide can include as little as 10 nucleotides, e.g., where the polynucleotide encodes an antigen. Furthermore, a “polynucleotide” can include both double- and single-stranded sequences and refers to, but is not limited to, cDNA from viral, prokaryotic or eukaryotic mRNA, genomic RNA and DNA sequences from viral (e.g. RNA and DNA viruses and retroviruses) or prokaryotic DNA, and also synthetic DNA sequences. The term also captures sequences that include any of the known base analogs of DNA and RNA. The term further includes modifications, such as deletions, additions and substitutions (eg. methylations or capping), to a native sequence, preferably such that the nucleic acid molecule encodes, for example, an antigenic protein. These modifications may be deliberate, as through site-directed mutagenesis, or through particular synthetic procedures, or through a genetic engineering approach, or may be accidental, such as through mutations of hosts, which produce the antigens. The terms “oligonucleotide” or “oligo” are used interchangeably herein.

The terms “porcine” and “swine” are used interchangeably and refer to any animal that is a member of the family Suidae such as, for example, a pig.

The term “protecting” refers to shielding eg. a mammal, in particular, a pig, from infection or a disease, by inducing an immune response to a particular pathogen, eg. circovirus. Such protection is generally achieved following treating a mammal with the vaccine compositions described herein, such as the chimeric PCV1-2 vaccine.

The terms “protein”, “polypeptide” and “peptide” refer to a polymer of amino acid residues and are not limited to a minimum length of the product. Thus, peptides, oligopeptides, dimers, multimers, and the like, are included within the definition. Both full-length proteins and fragments thereof are encompassed by the definition. The terms also include modifications, such as deletions, additions and substitutions (generally conservative in nature, but which may be non-conservative), to a native sequence, preferably such that the protein maintains the ability to elicit an immunological response within an animal to which the protein is administered. Also included are post-expression modifications, eg. glycosylation, acetylation, phosphorylation and the like.

In the present invention, “reducing mortality in pigs” refers to the ability of the vaccine or immunogenic composition, as described herein, to provide a significant decrease in the number of deaths associated with a pathogenic porcine circovirus. For example, under normal conditions, the percentage of deaths associated with porcine circovirus may be about 8-14% in an unvaccinated population of pigs. However, if the pig population had received a vaccine or immunogenic composition, as described herein, this percentage may drop to about 0.5 to 4% of the pig population. During an epidemic of porcine circovirus, 40% of the unvaccinated pig population may die after exposure to a pathogenic strain of porcine circovirus. However, if the pig population had been vaccinated with a vaccine or immunogenic composition, as described herein, this percentage would drop significantly, to about 10% of the pig population.

As used herein, the term “sequence homology” in all its grammatical forms refers to the relationship between proteins that possess a common evolutionary origin, including homologous proteins from different species (Reeck et al., 1987, Cell 50:667).

“SPF” refers to Specific-pathogen-free pigs.

Two DNA sequences are “substantially homologous” or “substantially similar” when at least about 75% (preferably at least about 80%, and more preferably at least about 90 or 95%, and most preferably about 99%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

Similarly, two amino acid sequences are “substantially homologous” or “substantially similar” when greater than 70% of the amino acids are identical, or functionally identical. Preferably, the similar or homologous sequences are identified by alignment using, for example, the GCG (Genetics Computer Group, Program Manual for the GCG Package, Version 7, Madison, Wis.) pileup program.

As used herein, “treatment” (including variations thereof, for example, “treat” or “treated”) refers to any one or more of the following: (i) the prevention of infection or reinfection, as in a traditional vaccine, (ii) the reduction in the severity of, or, in the elimination of symptoms, and (iii) the substantial or complete elimination of the pathogen or disorder in question. Hence, treatment may be effected prophylactically (prior to infection) or therapeutically (following infection). In the present invention, prophylactic treatment is the preferred mode. According to a particular embodiment of the present invention, compositions and methods are provided which treat, including prophylactically and/or therapeutically immunize, a host animal against a viral infection. The methods of the present invention are useful for conferring prophylactic and/or therapeutic immunity to a mammal, preferably a pig. The methods of the present invention can also be practiced on mammals for biomedical research applications.

The terms “vaccine” or “vaccine composition”, which are used interchangeably, refer to pharmaceutical compositions comprising at least one immunogenic composition that induces an immune response in an animal. A vaccine or vaccine composition may protect the animal from disease or possible death due to an infection, and may or may not include one or more additional components that enhance the immunological activity of the active component. A vaccine or vaccine composition may additionally comprise further components typical to pharmaceutical compositions. A vaccine or vaccine composition may additionally comprise further components typical to vaccines or vaccine compositions, including, for example, an adjuvant or an immunomodulator. The immunogenically active component of a vaccine may comprise complete live organisms in either their original form, or as attenuated organisms in a modified live vaccine, or organisms inactivated by appropriate methods in a killed or inactivated vaccine, or subunit vaccines comprising one or more immunogenic components of the virus, or genetically engineered, mutated or cloned vaccines prepared by methods known to those skilled in the art. A vaccine may comprise one or simultaneously more than one of the elements described above. In the present invention, the vaccine compositions include, but are not limited to, live, attenuated or killed/inactivated forms of whole chimeric porcine circoviruses, infectious nucleic acids encoding the chimeric porcine circoviruses, or other infectious DNA vaccines including plasmids, vectors, or other carriers to directly inject DNA into pigs.

“Virulence” is a measure of the severity of the disease caused by a microorganism. For example, in the present invention, “virulence” of a porcine circovirus may be measured or assessed by one or more of the following parameters: severity of clinical respiratory disease ranging from 0 (normal) to 6 (severe dyspnea and abdominal breathing) (Halbur et al, (1995), Vet. Pathol. 32:648-660); PCV2 DNA quantitation from one or more body fluids or tissues; histopathology findings, such as, but not limited to, measurement of the number and/or severity of microscopic lesions from one or more body tissues; for example, lung tissue may be scored for the presence and severity of interstitial pneumonia ranging from 0 (normal) to 6 (severe diffuse); or sections of heart, liver, kidney, ileum, and colon may be evaluated for the presence of lymphocytic inflammation and scored from 0 (none) to 3 (severe); or lymphoid tissue (including lymph nodes, tonsil or spleen) may be evaluated for the presence of lymphoid depletion ranging from 0 (normal) to 3 (severe) and histiocytic inflammation and replacement of follicles ranging from 0 (normal) to 3 (severe) (Opriessnig, et al. (2004) Vet. Pathol. 41:624-640). Virulence of a porcine circovirus strain may also be measured or assessed by its mortality rate in infected pigs. For example, certain strains of type 2B circovirus are known to exhibit a higher than average mortality rate, whereas the type 2A strains of porcine circovirus are generally known to exhibit a significantly lower mortality rate. In certain cases, these type 2B strains are also known to result in more severe microscopic lesions in the tissues of infected pigs, as compared to the less virulent type 2A strains. Accordingly, in the present invention, a “high virulence strain” or a “high mortality strain”, or a “high virulence/high mortality strain” refers to a strain of porcine circovirus that exhibits one or more of the above-noted characteristics or symptoms at a level significantly higher or greater than a low virulent/low mortality strain. In certain cases, the type 2B strains show higher mortality than the type 2A strains.

### General Description

Due to its potential impact on the pig industry, the development of a vaccine against pathogenic forms of porcine circovirus type 2 (PCV2) is of major importance. It is believed that the nonpathogenic PCV1 will be of limited use against PCV2 infections. Furthermore, pathogenic PCV2 strains, even if attenuated, are likely to be of limited value due to the usual tendency of a live virus to revert to its virulent state.

Moreover, new virulent strains of PCV2 have arisen, which are characterized in part by a higher than average mortality rate. These high virulence/high mortality pathogenic strains of PCV2 are designated PCV-2B, whereas the low virulence, low mortality pathogenic strains are designated PCV2A. Recently proposed alternate nomenclature for these two strains refers to the PCV2A strain as “Genotype II”, or “RFLP 422”, while the PCV2B strain is referred to as “Genotype I”, or “RFLP 321”. While certain of the previously described vaccine compositions may prove to be effective against the lower mortality, less virulent pathogenic strains of PCV2A, none have been shown to be effective against the high virulence pathogenic PCV2B strains, characterized in part by their higher than average mortality rates.

U.S. patent publications 20040253270 and 20030170270 describe a live, chimeric, nonpathogenic porcine circovirus, designated PCV1-2, for the inoculation of pigs against infection with PCV2 or PMWS caused by PCV2. It is constructed based upon the genomic backbone of the nonpathogenic PCV1 isolated by 1. Tischer et al. almost 30 years ago, but carries the immunogenic ORF2 capsid gene of the pathogenic PCV2. While this vaccine allows for the induction of an immune response against certain pathogenic, but low virulence/low mortality strains of PCV2A, the ability of this vaccine to protect pigs against high virulence/high mortality strains of PCV2B has not been shown until the present invention. Moreover, the ability to utilize an inactivated form of this chimeric porcine circovirus for cross-protection of pigs against the high virulence/high mortality strains of PCV2B has not been addressed previously. It is toward the use of an inactivated form of the chimeric PCV1-2 for eliciting a cross-protective immune response to a high virulence/high mortality strain of porcine circovirus type 2B that the present invention is directed.

Accordingly, the present invention relates to methods for immunizing a pig against a viral infection or postweaning multisystemic wasting syndrome (PMWS) caused by a pathogenic strain of porcine circovirus, or for reducing the rate of mortality associated with a high virulence/high mortality strain of porcine circovirus by administering to the pig a vaccine or immunogenic composition comprising an immunogenically effective amount of a type 1-type 2 chimeric porcine circovirus (PCV1-2) or the nucleic acid encoding the type 1-type 2 chimeric porcine circovirus.

In one embodiment of the present invention, the methods provide for immunizing a pig against a pathogenic porcine circovirus (PCV), which is a low virulence/low mortality type 2A strain (PCV2A).

In one embodiment, the methods provide for immunizing a pig against a pathogenic porcine circovirus, which is a high virulence/high mortality type 2B strain (PCV2B).

In particular, the methods of the present invention provide for the use of a vaccine or immunogenic composition comprising one or more of the following: 1) an avirulent/attenuated chimeric porcine circovirus; 2) a killed/inactivated chimeric porcine circovirus; 3) an avirulent, infectious chimeric DNA molecule, as described in U.S. patent publications 2003/0170270 and 2004/0253270 for immunizing pigs against a pathogenic circovirus infection. In one embodiment, the vaccine or immunogenic composition may comprise a PCV DNA vaccine (e.g. a plasmid vector expressing PCV2 ORF2 or chimeric PCV1-2). In one embodiment, the vaccine or immunogenic composition may comprise an inactivated viral vector (e.g. a baculovirus, adenovirus, or poxvirus, such as raccoonpox virus; or a bacterium, such as E. coli), that expresses PCV2 ORF2. In one embodiment, a vaccine or immunogenic composition wherein the ORF 2 gene is obtained from a type 2A strain of porcine circovirus may cross-protect against infections with a porcine type 2B, type 2C or type 2D strain, or any other variant. In one embodiment, a vaccine or immunogenic composition wherein the ORF 2 gene is obtained from a type 2B porcine circovirus may cross-protect against infections with a porcine type 2A, type 2C or type 2D strain, or any other variant. Moreover, in one embodiment, the vaccine or immunogenic composition used in the methods of the invention comprises an attenuated or an inactivated form of a type 1-type 2 chimeric porcine circovirus, PCV1-2. In one embodiment, the vaccine or immunogenic compositions comprise an avirulent, infectious chimeric DNA molecule of PCV1-2, which comprises a nucleic acid molecule encoding an infectious, nonpathogenic PCV-1. However, the immunogenic open reading frame 2 (ORF2) gene from the non-pathogenic PCV-1 strain, which encodes the viral capsid protein, was replaced by the open reading frame 2 (ORF2) gene from a pathogenic PCV-2 strain. The ORF2 gene that was utilized for preparation of the chimeric porcine circovirus vaccine was the ORF2 gene from a type 2A strain of porcine circovirus. The vaccine protected against pathogenic type 2A strains of PCV, wherein such strains contain the ORF2 capsid protein that is similar to the ORF2 gene utilized to make the chimeric PCV1-2 vaccine. Surprisingly, the vaccine was also shown to cross-protect against the more virulent, higher mortality strains of PCV2B.

Accordingly, both the infectious chimeric PCV1-2 DNA clone and the live, attenuated or killed/inactivated chimeric PCV1-2 circovirus contain the immunogenic capsid gene (ORF2) of the PCV-2 DNA cloned in the genomic backbone of the infectious, nonpathogenic PCV1 DNA clone. Generally, the capsid gene of the PCV-2 DNA replaces the ORF2 gene of the PCV-1 DNA in the nonpathogenic PCV-1 genomic structure, but it is contemplated that a variety of positional permutations may be constructed through genetic engineering to obtain other avirulent or attenuated chimeric DNA clones. While the vaccine or immunogenic composition comprising the chimeric PCV1-2 porcine circovirus protects pigs against infection with the pathogenic, PCV type 2A strain, it has never before been shown to be effective against the high virulence/high mortality type 2B strain, until described in the present invention. It is also contemplated that the vaccine or immunogenic compositions as described herein are effective for preventing one or more of the symptoms associated with postweaning multisystemic wasting syndrome (PMWS). These symptoms may include, for example, one or more of the following: respiratory disease, microscopic lesions in one or more tissues or organs, histiocytic inflammation, or lymphoid depletion. Moreover, the vaccines or immunogenic compositions described herein may be used with a second or third vaccine or immunogenic composition that protects pigs against one or more pathogenic porcine viruses or bacteria including: porcine reproductive and respiratory syndrome virus (PRRS), porcine parvovirus (PPV), Mycoplasma hyopneumoniae, Mycoplasma hyopneumoniae, Haemophilus parasuis, Pasteurella multocida, Streptococcum suis, Actinobacillus pleuropneumoniae, Bordetella bronchiseptica, Salmonella choleraesuis, Erysipelothrix rhusiopathiae, leptospira bacteria, swine influenza virus, Escherichia coli antigen, porcine respiratory coronavirus, rotavirus, a pathogen causative of Aujesky's Disease, and a pathogen causative of Swine Transmissible Gastroenteritis. For example, in one embodiment, the PCV vaccine or immunogenic composition may be combined with a porcine reproductive and respiratory syndrome virus (PRRS) vaccine or immunogenic composition. In one embodiment, the PCV vaccine or immunogenic composition may be combined with a Mycoplasma hyopneumoniae vaccine or immunogenic composition. In one embodiment, the PCV vaccine or immunogenic composition may be combined with a Mycoplasma hyopneumoniae vaccine or immunogenic composition and a porcine reproductive and respiratory syndrome virus (PRRS) vaccine or immunogenic composition.

### Use of the PCV1-2 Vaccines and Immunogenic Compositions

The present invention provides for the use of a vaccine or immunogenic composition comprising a chimeric PCV1-2 porcine circovirus for protection of pigs against viral infection and postweaning multisystemic wasting syndrome (PMWS).

The vaccine or immunogenic composition comprising the PCV1-2 chimeric porcine circovirus utilized in the present studies was prepared using the methods outlined by Meng, et al. in U.S. patent publications 2003/0170270 and 2004/0253270. In these publications, Meng et al. demonstrate that the chimeric PCV1-2 infectious DNA clone, having the immunogenic capsid gene (ORF2) of the pathogenic PCV-2 cloned into the nonpathogenic PCV-1 genomic backbone, induces a specific antibody response to the pathogenic PCV-2 capsid antigen while it uniquely retains the nonpathogenic nature of PCV-1 in pigs. Moreover, Meng et al. show that animals inoculated with the chimeric PCV1-2 infectious DNA clone develop a mild infection resembling that of PCV-1 inoculated animals while seroconverting to the antibody against the ORF2 capsid protein of the pathogenic PCV-2. The average length of viremia observed in PCV-1 and chimeric PCV1-2 inoculated animals was shorter, 0.625 weeks and 1 week respectively, than that in pathogenic PCV-2 inoculated animals, which was about 2.12 weeks. Furthermore, Meng et al. show that the lack of detectable chimeric PCV1-2 viremia in some inoculated animals does not affect seroconversion to antibody against PCV-2 ORF2 capsid protein in the PCV1-2 inoculated pigs. Their results indicate that, even though the chimeric PCV1-2 viremia is short or undetectable in some inoculated animals, the chimeric PCV1-2 virus is able to induce an antibody response against PCV-2 ORF2 capsid protein.

The inventors of the present application have conducted further studies with the chimeric porcine circovirus (PCV1-2), as described herein, and have shown that it is effective not only against the pathogenic type 2A porcine circovirus, but they have also shown that it is efficacious and shows cross-protection in pigs against the high virulence/high mortality type 2B strain(s) of porcine circovirus. Moreover, Meng et al. demonstrated that the live, attenuated PCV1-2 chimeric porcine circovirus vaccine provided protection against type 2A strains of porcine circovirus having the same ORF2 capsid protein as that present in the vaccine. The studies presented herein demonstrate that a vaccine or immunogenic composition comprising an inactivated form of the chimeric PCV1-2 porcine circovirus is effective against high virulence/high mortality type 2B strains, which have a different ORF2 capsid protein than the type 2A strains. These findings are of particular relevance given the fact that type 2A porcine circovirus appears to be present only in healthy pigs without clinical symptoms, while pigs exhibiting clinical symptoms of porcine circovirus infection are known to harbor both type 2A, as well as type 2B porcine circovirus.

In particular, the vaccine comprising PCV1-2, when administered as 1-shot to 3-4 week-old pigs, or as 2-shots at 3-4 weeks and 6-7 weeks of age, is able to prevent viremia associated with PCV-2 infection. Statistically significant differences were detected between the groups that received either one dose of the composition (Group 1), or two doses of the composition (Group 2) prior to challenge, and the Group that did not receive the vaccine composition prior to challenge (Group 3) at days 7, 14 and 21 post infection (PI).

At necropsy, the number of gross lesions did not allow for evaluation of the effect of the cPCV1-2 vaccine, since very few pigs presented gross lesions in all groups examined, and those lesions observed could be also, in some cases, attributed to other pathologies.

However, at microscopic level, the development of lesions (mainly in lymphoid tissues) typical of PCV-2 infection were reduced in vaccinated animals: in the non-vaccinated and challenged group, 38.09% of the pigs presented mild lymphocyte depletion and infiltration, while in the vaccinated and challenged groups (1-shot and 2-shots), these were only observed in one pig of each group (5.88 and 7.14%, respectively).

The presence of the PCV-2 genome in target tissues was detected by in situ hybridization (ISH) in 33.3% of the non-vaccinated and challenged pigs. In contrast, none of vaccinated and challenged pigs had PCV2 nucleic acid within tissues.

The inventors have thus demonstrated that a killed and adjuvanted vaccine or immunogenic composition comprising the type 1-type 2 chimeric porcine circovirus (PCV1-2) is effective in protecting pigs against the adverse effects of PCV-2 infection, including PCV-2 viremia, lymphoid tissue lesions and the presence of the PCV-2 genome in tissues, even when administered 4 months prior to challenge.

However, the inventors also demonstrated the ability of the PCV1-2 vaccine to protect against pathogenic type 2A strains, as well as, to provide cross-protection against the high virulence/high mortality type 2B European strains of porcine circovirus. The results of these studies are presented in greater detail in the Examples to follow.

### Nucleic Acids of the Invention

The purified and isolated nucleic acid molecules as described herein for preparation of the vaccine or immunogenic compositions comprise the full-length DNA sequence of the cloned chimeric PCV1-2 DNA as set forth in SEQ ID NO: 1, which was deposited in the American Type Culture Collection under Patent Deposit Designation PTA-3912 (see Meng et al., U.S. patent publication number 2003/0170270 and 2004/0253270); its complementary strand (i.e., reverse and opposite base pairs) or nucleotide sequences having at least 80% homology, more preferably about 95 to 99% homology, to the chimeric nucleotide sequence (i.e., a significant active portion of the whole gene). Conventional methods that are well known in the art can be used to make the complementary strands or the nucleotide sequences possessing high homology, for instance, by the art-recognized standard or high stringency hybridization techniques. The purified and isolated nucleic acid molecule comprising the DNA sequence of the immunogenic capsid gene of the cloned chimeric PCV1-2 DNA is set forth in SEQ ID NO: 3.

Accordingly, any suitable animal cell containing the chimeric PCV1-2 nucleic acid molecule described herein can produce live, infectious porcine circoviruses. The live, infectious chimeric virus is derived from the chimeric DNA clone by transfecting, for example, PK-15 cells via in vitro or in vivo. As noted above, one example of the cloned chimeric PCV1-2 DNA is the nucleotide sequence set forth in SEQ ID NO: 1. The invention further envisions that the chimeric virus may be derived from the complementary strand or a nucleotide sequence having high homology, at least 80%, and more preferably, 95-99% homology, to the chimeric nucleotide sequence.

Also included within the scope of the present invention are biologically functional plasmids, viral vectors and the like that contain the chimeric nucleic acid molecules described herein, suitable host cells transfected by the vectors comprising the chimeric DNA clones and the immunogenic polypeptide expression products. In one embodiment, the immunogenic protein is the capsid protein encoded by ORF2 from a type 2A strain of porcine circovirus. The amino acid sequence of this capsid protein in the chimeric porcine circovirus is set forth in SEQ ID NO: 4. Biologically active variants thereof are further encompassed by the invention. One of ordinary skill in the art would know how to modify, substitute, delete, etc., amino acid(s) from the polypeptide sequence and produce biologically active variants that retain the same, or substantially the same, activity as the parent sequence without undue effort.

To produce the immunogenic polypeptide products of this invention, the process may include the following steps: growing, under suitable nutrient conditions, prokaryotic or eucaryotic host cells transfected with the chimeric nucleic acid molecules described herein in a manner that allows for expression of the polypeptide products, and isolating the desired polypeptide products by standard methods known in the art. It is contemplated that the immunogenic proteins may be prepared by other techniques such as, for example, biochemical synthesis and the like.

### Vaccines and Immunogenic Compositions

The preparation of vaccines or immunogenic compositions comprising the chimeric PCV1-2 viral clones, and methods of using them for protection against high virulence/high mortality strains of porcine circovirus, are also included within the scope of the present invention. Inoculated pigs are protected from serious viral infection and PMWS caused by PCV2, type 2A and type 2B. The method protects pigs in need of protection against viral infection or PMWS by administering to the pig an immunogenically effective amount of a vaccine according to the invention, such as, for example, a vaccine comprising an immunogenic amount of the chimeric PCV1-2 DNA, the cloned chimeric virus, a plasmid or viral vector containing the chimeric DNA of PCV1-2, the polypeptide expression products, etc. The vaccine as described herein may be administered with a second or third vaccine or immunogenic composition against other porcine pathogens, including for example, PRRSV, PPV, and other infectious swine agents selected from the following: Mycoplasma hyopneumoniae, Haemophilus parasuis, Pasteurella multocida, Streptococcum suis, Actinobacillus pleuropneumoniae, Bordetella bronchiseptica, Salmonella choleraesuis, Erysipelothrix rhusiopathiae, leptospira bacteria, swine influenza virus, porcine parvovirus, Escherichia coli, porcine respiratory coronavirus, rotavirus, a pathogen causative of Aujesky's Disease, and a pathogen causative of Swine Transmissible Gastroenteritis antigen. Particular combinations may include a PCV vaccine or immunogenic composition in combination with a PRRSV vaccine or immunogenic composition; a PCV vaccine or immunogenic composition in combination with a Mycoplasma hyopneumoniae vaccine or immunogenic composition; or a PSV vaccine or immunogenic in combination with both of the foregoing vaccines or immunogenic compositions. Immune stimulants may be given concurrently to the pig to provide a broad spectrum of protection against other viral or bacterial infections.

The vaccines or immunogenic compositions used in the methods of the invention are not restricted to any particular type or method of preparation. The vaccines or immunogenic compositions may include, for example, a nucleic acid encoding one or more of the porcine circovirus proteins, infectious DNA vaccines (ie. using plasmids, vectors, or other conventional carriers to directly inject DNA into pigs), live vaccines, modified live vaccines, inactivated vaccines, subunit vaccines, attenuated vaccines, genetically engineered vaccines, etc. In certain embodiments, the vaccine may include the infectious chimeric PCV1-2 (cPCV1-2) DNA, the cloned PCV chimeric DNA genome in suitable plasmids or vectors such as, for example, the pSK vector, an avirulent, live chimeric virus, an inactivated chimeric virus, etc., or a viral vector may be used, such as, but not limited to, a baculovirus vector, an adenovirus vector, or a poxvirus vector, such as raccoonpox virus, or a bacterial vector, such as E. coli. Any of the above may be used in combination with a nontoxic, physiologically acceptable carrier and, optionally, one or more adjuvants.

The PCV1-2 chimeric porcine circovirus of the present invention overcomes certain disadvantages associated with live viral vaccines, such as the potential risk of contamination with live adventitious viral agents or the risk of the virus reverting to a more virulent form in the field. The initial chimeric PCV1-2 porcine circovirus was constructed using the backbone of the non-pathogenic PCV-1 and only the immunogenic genes of the pathogenic PCV2. Thus, the chimeric DNA constructs a live, replicating chimeric virus that is nonpathogenic yet elicits the complete, beneficial immune responses of live viral vaccines against the pathogenic PCV2 virus. The live virus vaccine based on the chimeric virus will have little chance, if any, for reversion to a pathogenic phenotype. Thus, the new chimeric virus based on the structure of the nonpathogenic PCV1 has a huge advantage over any recombinant PCV2 DNA virus, any live, attenuated PCV2 vaccine or any other type of vaccine predicated solely on PCV2 for immunity against the PCV2 infections. Moreover, the present invention provides evidence that the chimeric PCV1-2 porcine circovirus, when inactivated, also provides protection against not only pathogenic type 2A porcine circoviruses, but also provides protection against the high virulence/high mortality type 2B strains of porcine circovirus. To prepare an inactivated virus vaccine, for instance, the virus propagation from the infectious DNA clone is done by methods known in the art or described herein. Serial virus inactivation is then optimized by protocols generally known to those of ordinary skill in the art.

Inactivated virus vaccines or immunogenic compositions may be prepared by treating the chimeric virus derived from the cloned PCV DNA with inactivating agents such as formalin or hydrophobic solvents, acids, etc., by irradiation with ultraviolet light or X-rays, by heating, etc. Inactivation is conducted in a manner understood in the art. For example, in chemical inactivation, a suitable virus sample or serum sample containing the virus is treated for a sufficient length of time with a sufficient amount or concentration of inactivating agent at a sufficiently high (or low, depending on the inactivating agent) temperature or pH to inactivate the virus. Inactivation by heating is conducted at a temperature and for a length of time sufficient to inactivate the virus. Inactivation by irradiation is conducted using a wavelength of light or other energy source for a length of time sufficient to inactivate the virus. The virus is considered inactivated if it is unable to infect a cell susceptible to infection.

The preparation of subunit vaccines typically differs from the preparation of a modified live vaccine or an inactivated vaccine. Prior to preparation of a subunit vaccine, the protective or antigenic components of the vaccine must be identified. Such protective or antigenic components include certain amino acid segments or fragments of the viral capsid proteins which raise a particularly strong protective or immunological response in pigs; single or multiple viral capsid proteins themselves, oligomers thereof, and higher-order associations of the viral capsid proteins which form virus substructures or identifiable parts or units of such substructures; oligoglycosides, glycolipids or glycoproteins present on or near the surface of the virus or in viral substructures such as the lipoproteins or lipid groups associated with the virus, etc. Preferably, a capsid protein, such as the protein encoded by the ORF2 gene, is employed as the antigenic component of the subunit vaccine. Other proteins encoded by the infectious DNA clone may also be used. These immunogenic components are readily identified by methods known in the art. Once identified, the protective or antigenic portions of the virus (i.e., the “subunit”) are subsequently purified and/or cloned by procedures known in the art. The subunit vaccine provides an advantage over other vaccines based on the live virus since the subunit, such as highly purified subunits of the virus, is less toxic than the whole virus.

If the subunit vaccine is produced through recombinant genetic techniques, expression of the cloned subunit such as the ORF2 (capsid) gene, for example, may be optimized by methods known to those in the art (see, for example, Maniatis et al., “Molecular Cloning: A Laboratory Manual,” Cold Spring Harbor Laboratory, Cold Spring Harbor, Mass., 1989). If the subunit being employed represents an intact structural feature of the virus, such as an entire capsid protein, the procedure for its isolation from the virus must then be optimized. In either case, after optimization of the inactivation protocol, the subunit purification protocol may be optimized prior to manufacture.

To prepare attenuated vaccines from pathogenic clones, the tissue culture adapted, live, pathogenic PCV2 is first attenuated (rendered nonpathogenic or harmless) by methods known in the art, typically made by serial passage through cell cultures. Attenuation of pathogenic clones may also be made by gene deletions or viral-producing gene mutations. Then, the attenuated PCV2 viruses may be used to construct additional chimeric PCV1-2 viruses that retain the nonpathogenic phenotype of PCV1 but can vary in the strength of the immunogenicity traits selected from the PCV2 genome through recombinant technology.

Advantageously, the live chimeric PCV1-2 virus is naturally avirulent when constructed through genetic engineering, and it does not require time-consuming attenuation procedures. The virus uniquely serves as a live but nonpathogenic replicating virus that produces immunogenic proteins against PCV2 during virus replication, which can then elicit a full range of immune responses against the pathogenic PCV2. Moreover, the present invention provides further unexpected results in that an inactivated form of the chimeric PCV1-2 also provides protection against both type 2A pathogenic porcine circovirus, as well as against the high virulence/high mortality type 2B porcine circoviruses.

Another preferred vaccine of the present invention utilizes suitable plasmids for delivering the nonpathogenic chimeric DNA clone to pigs. In contrast to the traditional vaccine that uses live or killed cell culture propagated whole virus, this invention provides for the direct inoculation of pigs with the plasmid DNA containing the infectious chimeric viral genome.

Additional genetically engineered vaccines, which are desirable in the present invention, are produced by techniques known in the art. Such techniques involve, but are not limited to, further manipulation of recombinant DNA, modification of or substitutions to the amino acid sequences of the recombinant proteins and the like.

Genetically engineered vaccines based on recombinant DNA technology are made, for instance, by identifying alternative portions of the viral gene encoding proteins responsible for inducing a stronger immune or protective response in pigs (e.g., proteins derived from ORF3, ORF4, etc.). Such identified genes or immuno-dominant fragments can be cloned into standard protein expression vectors, such as the baculovirus vector, and used to infect appropriate host cells (see, for example, O'Reilly et al., “Baculovirus Expression Vectors: A Lab Manual,” Freeman & Co., 1992). The host cells are cultured, thus expressing the desired vaccine proteins, which can be purified to the desired extent and formulated into a suitable vaccine product.

If the clones retain any undesirable natural abilities of causing disease, it is also possible to pinpoint the nucleotide sequences in the viral genome responsible for the virulence, and genetically engineer the virus avirulent through, for example, site-directed mutagenesis. Site-directed mutagenesis is able to add, delete or change one or more nucleotides (see, for instance, Zoller et al., DNA 3:479-488, 1984). An oligonucleotide is synthesized containing the desired mutation and annealed to a portion of single stranded viral DNA. The hybrid molecule, which results from that procedure, is employed to transform bacteria. Then double-stranded DNA, which is isolated containing the appropriate mutation, is used to produce full-length DNA by ligation to a restriction fragment of the latter that is subsequently transfected into a suitable cell culture. Ligation of the genome into the suitable vector for transfer may be accomplished through any standard technique known to those of ordinary skill in the art. Transfection of the vector into host cells for the production of viral progeny may be done using any of the conventional methods such as calcium-phosphate or DEAE-dextran mediated transfection, electroporation, protoplast fusion and other well-known techniques (e.g., Sambrook et al., “Molecular Cloning: A Laboratory Manual,” Cold Spring Harbor Laboratory Press, 1989). The cloned virus then exhibits the desired mutation. Alternatively, two oligonucleotides can be synthesized which contain the appropriate mutation. These may be annealed to form double-stranded DNA that can be inserted in the viral DNA to produce full-length DNA.

Genetically engineered proteins, useful in vaccines, for instance, may be expressed in insect cells, yeast cells or mammalian cells. The genetically engineered proteins, which may be purified or isolated by conventional methods, can be directly inoculated into pigs to confer protection against viral infection or postweaning multisystemic wasting syndrome (PMWS) caused by PCV2.

An insect cell line (like HI-FIVE) can be transformed with a transfer vector containing nucleic acid molecules obtained from the virus or copied from the viral genome which encodes one or more of the immuno-dominant proteins of the virus. The transfer vector includes, for example, linearized baculovirus DNA and a plasmid containing the desired polynucleotides. The host cell line may be co-transfected with the linearized baculovirus DNA and a plasmid in order to make a recombinant baculovirus.

Alternatively, DNA from a pig suffering from PMWS, which encode one or more capsid proteins, the infectious PCV2 molecular DNA clone or the cloned PCV chimeric DNA genome can be inserted into live vectors, such as a poxvirus or an adenovirus and used as a vaccine.

An immunogenically effective amount of the vaccines of the present invention is administered to a pig in need of protection against viral infection or PMWS. The immunogenically effective amount or the immunogenic amount that inoculates the pig can be easily determined or readily titrated by routine testing. An effective amount is one in which a sufficient immunological response to the vaccine is attained to protect the pig exposed to the virus which causes PMWS. Preferably, the pig is protected to an extent in which one to all of the adverse physiological symptoms or effects of the viral disease are significantly reduced, ameliorated or totally prevented.

The vaccine or immunogenic composition can be administered in a single dose or in repeated doses. Dosages may range, for example, from 50 to 5,000 micrograms of the plasmid DNA containing the infectious chimeric DNA genome (dependent upon the concentration of the immuno-active component of the vaccine), but should not contain an amount of virus-based antigen sufficient to result in an adverse reaction or physiological symptoms of viral infection. Methods are known in the art for determining or titrating suitable dosages of active antigenic agent based on the weight of the pig, concentration of the antigen and other typical factors. Preferably, the infectious chimeric viral DNA clone is used as a vaccine, or a live infectious chimeric virus can be generated in vitro and then the live chimeric virus is used as a vaccine. In that case, 100 to 200 micrograms of cloned chimeric PCV DNA or about 10,000 50% tissue culture infective dose (TCID₅₀) of live chimeric virus can be given to a pig.

Desirably, the vaccine or immunogenic composition is administered to a pig not yet exposed to the PCV virus. The vaccine containing the chimeric PCV1-2 infectious DNA clone or other antigenic forms thereof can conveniently be administered intranasally, transdermally (i.e., applied on or at the skin surface for systemic absorption), parenterally, etc. The parenteral route of administration includes, but is not limited to, intramuscular, intravenous, intraperitoneal, intradermal (i.e., injected or otherwise placed under the skin) routes and the like. Since the intramuscular and intradermal routes of inoculation have been successful in other studies using viral infectious DNA clones (E. E. Sparger et al., “Infection of cats by injection with DNA of feline immunodeficiency virus molecular clone,” Virology 238:157-160 (1997); L. Willems et al., “In vivo transfection of bovine leukemia provirus into sheep,” Virology 189:775-777 (1992)), these routes are most preferred, in addition to the practical intranasal route of administration. Although less convenient, it is also contemplated that the vaccine is given to the pig through the intralymphoid route of inoculation. A unique, highly preferred method of administration involves directly injecting the plasmid DNA containing PCV1-2 chimera or the chimeric PCV1-2 virus (attenuated or inactivated) into the pig intramuscularly, intradermally, intralymphoidly, etc.

When administered as a liquid, the present vaccine may be prepared in the form of an aqueous solution, syrup, an elixir, a tincture and the like. Such formulations are known in the art and are typically prepared by dissolution of the antigen and other typical additives in the appropriate carrier or solvent systems. Suitable “physiologically acceptable” carriers or solvents include, but are not limited to, water, saline, ethanol, ethylene glycol, glycerol, etc. Typical additives are, for example, certified dyes, flavors, sweeteners and antimicrobial preservatives such as thimerosal (sodium ethylmercurithiosalicylate). Such solutions may be stabilized, for example, by addition of partially hydrolyzed gelatin, sorbitol or cell culture medium, and may be buffered by conventional methods using reagents known in the art, such as sodium hydrogen phosphate, sodium dihydrogen phosphate, potassium hydrogen phosphate, potassium dihydrogen phosphate, a mixture thereof, and the like.

Liquid formulations also may include suspensions and emulsions that contain suspending or emulsifying agents in combination with other standard co-formulants. These types of liquid formulations may be prepared by conventional methods. Suspensions, for example, may be prepared using a colloid mill. Emulsions, for example, may be prepared using a homogenizer.

Parenteral formulations, designed for injection into body fluid systems, require proper isotonicity and pH buffering to the corresponding levels of porcine body fluids. Isotonicity can be appropriately adjusted with sodium chloride and other salts as needed. Suitable solvents, such as ethanol or propylene glycol, can be used to increase the solubility of the ingredients in the formulation and the stability of the liquid preparation. Further additives that can be employed in the present vaccine include, but are not limited to, dextrose, conventional antioxidants and conventional chelating agents such as ethylenediamine tetraacetic acid (EDTA). Parenteral dosage forms must also be sterilized prior to use.

Methods of preparing an infectious, nonpathogenic chimeric nucleic acid molecule of PCV1-2 are described herein. These methods include removing an open reading frame (ORF) gene of a nucleic acid molecule encoding an infectious nonpathogenic PCV1, replacing the same position with an immunogenic ORF gene of a nucleic acid molecule encoding an infectious pathogenic PCV2, and recovering the chimeric nucleic acid molecule. The nucleic acid molecule is typically DNA. A preferred method replaces the ORF2 gene of the nonpathogenic PCV1 DNA with the immunogenic ORF2 capsid gene of the infectious pathogenic molecular DNA of PCV2 described herein. It is contemplated that other ORF positions or immunogenic fragments thereof can be exchanged between the PCV1 and PCV2 DNA to construct the attenuated infectious chimeric DNA clones according to the methods described herein.

The recombinant nucleic acid molecule is then used to construct the live, infectious, replicating chimeric virus of the present invention that advantageously retains the nonpathogenic nature of PCV1 yet expresses the immunogenic ORF2 protein of the pathogenic PCV2 and elicits a complete immune response against the pathogenic PCV2. Desirably, the PCV1-2 DNA clone serves as a genetically engineered avirulent, live vaccine against PCV2 infection and PMWS in pigs.

As described herein, the immunogenic ORF2 capsid gene is switched between the pathogenic PCV2 and the nonpathogenic PCV1 to produce the unique structure of the chimeric PCV1-2 infectious DNA clone. The chimeric PCV1-2 infectious clone replicated, expressed the immunogenic ORF2 capsid antigen in vitro and in vivo, and induced a specific antibody response against PCV2 ORF2 but retained the nonpathogenic nature of PCV1. The chimeric PCV1-2 infectious DNA clone has the ability to induce a strong immune response against PCV2 while inducing only a limited infection with mild pathologic lesions similar to that of the nonpathogenic PCV1. For vaccine development, the relatively easy storage and stability of cloned DNA, and the economy of large-scale recombinant PCV2 plasmid DNA and chimeric PCV1-2 DNA clone production provides an attractive means of delivering a live, infectious viral DNA vaccine or genetically engineered, attenuated viral vaccines to pigs. Therefore, the chimeric PCV1-2 infectious DNA clone or a chimeric PCV1-2 virus as described herein is a useful vaccine candidate against PCV2 infection and PMWS.

The infectious PCV1/PCV2 chimeric DNA clone (strain designation “PCV1-2 chimera”), the infectious PCV2 molecular DNA clone (strain designation “PCV2 clone”) and the biologically pure and homogeneous PCV2 stock derived from an Iowa sample of PCV2 that had been isolated from a pig with severe PMWS and identified as isolate number 40895 (strain designation “PCV2 #40895”) are deposited under the conditions mandated by 37 C.F.R. §1.808 and maintained pursuant to the Budapest Treaty in the American Type Culture Collection (ATCC), 10801 University Boulevard, Manassas, Va. 20110-2209, U.S.A. The DNA sequences described herein are contained within 6,490 bp plasmids cloned into pBluescript SK(+) vector (pSK) (Stratagene Inc., La Jolla, Calif.) and transformed into Escherichia coli DH5a competent cells. The plasmids containing the infectious chimeric PCV1-2 DNA clone (identified as “chimeric porcine circovirus Type 1 (PCV1) and Type 2 (PCV2) infectious DNA clone”) and the infectious PCV2 molecular DNA clone (identified as “infectious DNA clone of Type 2 porcine circovirus (PCV2)”) have been deposited in the ATCC on Dec. 7, 2001 and have been assigned ATCC Patent Deposit Designations PTA-3912 and PTA-3913, respectively. It should be appreciated that other plasmids, which may be readily constructed using site-directed mutagenesis and the techniques described herein, are also encompassed within the scope of the present invention. The biologically pure and homogeneous PCV2 sample of isolate number 40895 (identified as “Type 2 porcine circovirus (PCV2)”) has also been deposited in the ATCC on Dec. 7, 2001 and has been assigned ATCC Patent Deposit Designation PTA-3914. The genomic (nucleotide) sequence of the PCV2 isolate number 40895 has been deposited with the Genbank database and has been publicly available since Jul. 23, 2000 under accession number AF264042. The chimeric PCV1-2 vaccine is manufactured by Fort Dodge Animal Health, Iowa and is available as Suvaxyn® PCV One-Dose.

### Adjuvants

The live, attenuated chimeric porcine circovirus, or the killed/inactivated chimeric porcine circovirus, or the nucleic acid encoding the chimeric porcine circovirus, or the plasmid or viral vector into which the ORF gene from PCV has been incorporated may be delivered with or without an adjuvant. In one embodiment, the vaccine is a killed/inactivated chimeric PCV1-2 circovirus, which is administered with an adjuvant. An adjuvant is a substance that increases the immunological response of the pig to the vaccine. The adjuvant may be administered at the same time and at the same site as the vaccine, or at a different time, for example, as a booster. Adjuvants also may advantageously be administered to the pig in a manner or at a site different from the manner or site in which the vaccine is administered. Suitable adjuvants include, but are not limited to, aluminum hydroxide (alum), immunostimulating complexes (ISCOMS), non-ionic block polymers or copolymers, cytokines (like IL-1, IL-2, IL-7, IFN-α, IFN-β, IFN-γ, etc.), saponins, monophosphoryl lipid A (MLA), muramyl dipeptides (MDP) and the like. Other suitable adjuvants include, for example, aluminum potassium sulfate, heat-labile or heat-stable enterotoxin isolated from Escherichia coli, cholera toxin or the B subunit thereof, diphtheria toxin, tetanus toxin, pertussis toxin, Freund's incomplete or complete adjuvant, etc. Toxin-based adjuvants, such as diphtheria toxin, tetanus toxin and pertussis toxin may be inactivated prior to use, for example, by treatment with formaldehyde.

### Assays for Measuring Immune Responses

The functional outcome of vaccinating a pig against porcine circovirus can be assessed by suitable assays that monitor induction of cellular or humoral immunity or T cell activity. These assays are known to one skilled in the art, but may include measurement of cytolytic T cell activity using for example, a chromium release assay. Alternatively, T cell proliferative assays may be used as an indication of immune reactivity or lack thereof. In addition, in vivo studies can be done to assess the level of protection in a mammal vaccinated against a pathogen using the methods of the present invention. Typical in vivo assays may involve vaccinating an animal with an antigen, such as the chimeric porcine circovirus described herein. After waiting for a time sufficient for induction of an antibody or T cell response to occur, generally from about one to two weeks after injection, the animals will be challenged with the antigen, such as either a virus, and amelioration of one or more symptoms associated with the viral infection, or survival of the animals is monitored. A successful vaccination regimen against porcine circovirus will result in significant decrease in one or more symptoms associated with the viral infection, or a decrease in viremia, or a decrease in the number or severity of lesions associated with a viral infection, or survival when compared to the non-vaccinated controls. Serum may also be collected to monitor levels of antibodies generated in response to the vaccine injections, as measured by methods known to those skilled in the art.

### Methods for Comparing Porcine Circovirus Type 2A and Type 2B Isolates

One of the primary advantages of the methods of the present invention relates to the ability of the inactivated and adjuvanted chimeric PCV1-2 porcine circovirus vaccine or immunogenic composition to induce an immune response that protects against not only the pathogenic type 2A porcine circovirus, but it also cross-protects against the high virulence/high mortality porcine circovirus type 2B strains.

The type 2A and type 2B strains may be differentiated through use of restriction fragment length polymorphism (RFLP) analysis. RFLP uses enzyme digestion of viral nucleic acid (partial or whole), which results in a specific cutting pattern that is visualized on a gel. If there are differences between viruses at the site of enzyme cutting, different patterns can be observed. This fingerprinting technique has been commonly used for DNA viruses. Meng et al. (U.S. patent publication 2005/0147966) describe the use of a PCR-RFLP assay using the NcoI restriction enzyme to distinguish between non-pathogenic type 1 porcine circoviruses and pathogenic type 2 porcine circoviruses. An ORF2 based PCR-RFLP assay described in 2000 using HinfI, HinP1I, KpnI, MseI, and RsaI enzymes is able to distinguish among PCV2 isolates (PCV2A, B, C, D, and E) (Hamel A L, Lin L L, Sachvie C, Grudeski E, Nayar G P: PCR detection and characterization of type-2 porcine circovirus. Can J Vet Res. 64:44-52, 2000).

An ORF2 based PCR-RFLP assay using Sau3AI, BanII, NspI, XbaI, and CfrI enzymes has been described recently and is able to distinguish 9 different PCV2 genotypes (Wen L, Guo X, Yang H: Genotyping of porcine circovirus type 2 from a variety of clinical conditions in China. Vet Microbiol. 110:141-146, 2005). PCV2 RFLP analysis showed that there was a significant change from RFLP type 422 to type 321 in 2005 in Ontario, Canada (Delay J, McEwen B, Carman S, van Dreuel T, Fairles J: Porcine circovirus type 2-associated disease is increasing. AHL Newsletter. 9:22, 2005).

In addition to using RFLP analysis to differentiate between type 2A and type 2B porcine circoviruses, it is believed that these two strains may be differentiated on the basis of sequences analysis.

For example, with sequence analysis it is possible to characterize the genetic information and compare isolates to each other (Choi J, Stevenson G W, Kiupel M, Harrach B, Anothayanontha L, Kanitz C L, Mittal SK: Sequence analysis of old and new strains of porcine circovirus associated with congenital tremors in pigs and their comparison with strains involved with postweaning multisystemic wasting syndrome. Can J Vet Res. 66:217-224, 2002; De Boisseson C, Beven V, Bigarre L, Thiery R, Rose N, Eveno E, Madec F, Jestin A: Molecular characterization of porcine circovirus type 2 isolates from post-weaning multisystemic wasting syndrome-affected and non-affected pigs. J Gen Virol. 85:293-304, 2004; Fenaux M, Halbur P G, Gill M, Toth T E, Meng X J: Genetic characterization of type 2 porcine circovirus (PCV-2) from pigs with postweaning multisystemic wasting syndrome in different geographic regions of North America and development of a differential PCR-restriction fragment length polymorphism assay to detect and differentiate between infections with PCV-1 and PCV-2. J Clin Microbiol. 38:2494-2503, 2000; Grierson S S, King D P, Sandvik T, Hicks D, Spencer Y, Drew T W, Banks M: Detection and genetic typing of type 2 porcine circovirus in archived pig tissues from the UK. Arch Viroi. 149:1171-1183, 2004; Kim J H, Lyoo YS: Genetic characterization of porcine circovirus-2 field isolates from PMWS pigs. J Vet Sci. 3:31-39, 2002; Mankertz A, Domingo M, Folch J M, LeCann P, Jestin A, Segales J, Chmielewicz B, Plana-Duran J, Soike D: Characterisation of PCV-2 isolates from Spain, Germany and France. Virus Res. 66:65-77, 2000). To further investigate possible differences among PCV2 isolates it is possible to sequence the entire PCV2 genome or to sequence only ORF2.

The two strains also differ with respect to the pathology, clinical symptoms and mortality associated with the disease itself, with type 2A demonstrating less severe lesions in bodily tissues and a lower mortality rate, as compared to the more severe lesions and higher mortality rate associated with type 2B strains. These clinical parameters may be measured using standard procedures known in the art and as demonstrated in the present invention.

### EXAMPLES

The following examples demonstrate certain aspects of the present invention. However, it is to be understood that these examples are for illustration only and do not purport to be wholly definitive as to conditions and scope of this invention. It should be appreciated that when typical reaction conditions (e.g., temperature, reaction times, etc.) have been given, the conditions both above and below the specified ranges can also be used, though generally less conveniently. All parts and percents referred to herein are on a weight basis and all temperatures are expressed in degrees centigrade unless otherwise specified.

### Example 1

### Construction of the PCV2 Infectious DNA Clone

The procedure for construction of the PCV2 Infectious DNA clone is described in Meng et al., U.S. patent publications 2003/0170270 and 2004/0253270. Briefly, a pair of PCR primers was designed according to the published sequence of the PCV2 isolate 40895 (Fenaux M, Halbur P G, Gill M, Toth T E, Meng X J: Genetic characterization of type 2 porcine circovirus (PCV-2) from pigs with postweaning multisystemic wasting syndrome in different geographic regions of North America and development of a differential PCR-restriction fragment length polymorphism assay to detect and differentiate between infections with PCV-1 and PCV-2. J Clin Microbiol. 38:2494-2503, 2000): forward primer F-PCVSAC2 (5′-GAACCGCGGGCTGGCTGMCTTTTGAAAGT-3′), set forth in SEQ ID NO:19, and reverse primer R-PCVSAC2 (5′-GCACCGCGGAAATTTCTGACAAA CGTTACA-3′), set forth in SEQ ID NO:20. This pair of primers amplifies the complete genome of PCV2 with an overlapping region containing the unique SacI restriction enzyme site. DNA was extracted using the QIAamp DNA Minikit (Qiagen, Inc., Valencia, Calif.) from a spleen tissue sample of a pig with naturally occurring PMWS (isolate 40895) (M. Fenaux et al., 2000, supra). The extracted DNA was amplified by PCR with AmpliTaq Gold polymerase (Perkin-Elmer, Norwalk, Conn.). The PCR reaction consisted of an initial enzyme activation step at 95° C. for 9 min, followed by 35 cycles of denaturation at 94° C. for 1 min, annealing at 48° C. for 1 min, extension at 72° C. for 3 min, and a final extension at 72° C. for 7 min. The PCR product of expected size was separated by gel electrophoresis and purified with the glassmilk procedure with a Geneclean Kit (Bio 101, Inc., La Jolla, Calif.).

To construct a molecular DNA clone containing a tandem dimer of PCV2 genome, the PCR product containing the complete PCV2 genome was first ligated into the advanTAge plasmid vector (Clontech, Palo Alto, Calif.). E. Coli DH5.alpha. competent cells were transformed. The recombinant plasmids were verified by restriction enzyme digestion. The full length PCV2 genomic DNA was excised from the advanTAge vector by digestion with SacI restriction enzyme. The digested PCV2 genomic DNA was ligated with T4 DNA ligase at 37° C. for only 10 min, which favors the production of tandem dimers. The tandem dimers were subsequently cloned into pBluescript SK(+) vector (pSK) (Stratagene Inc., La Jolla, Calif.). Recombinant plasmids containing tandem dimers of PCV2 genome (herein referred to as PCV2 molecular DNA clone) were confirmed by PCR, restriction enzyme digestion, and DNA sequencing. The DNA concentration of the recombinant plasmids was determined spectrophotometrically.

Specifically, the complete genome of the PCV2 (isolate 40895) was amplified by PCR to construct the infectious PCV2 molecular DNA clone. Two copies of the complete PCV2 genome were ligated in tandem into the pSK vector to produce the PCV2 molecular DNA clone. The infectivity of the PCV2 molecular DNA clone was determined by in vitro transfection of the PK-15 cells. IFA with PCV2-specific antibody confirmed that the molecular DNA clone is infectious in vitro and that about 10-15% of the PK-15 cells were transfected. PCV2-specific antigen was visualized by IFA in the nucleus, and to a lesser degree, cytoplasm of the transfected cells. The cells mock-transfected with the empty pSK vector remained negative for PCV2 antigen.

### Example 2

### In Vitro Transfection with the PCV2 Molecular DNA Clone and Generation of a Biologically Pure and Homogenous PCV2 Infectious Virus Stock

The method for testing the PCV2 molecular clone and for generation of a biologically pure and homogeneous PCV2 infectious virus stock is also described in Meng et al. (U.S. patent publications 2003/0170270 and 2004/0253270). Briefly, PK-15 cells free of PCV1 contamination were grown in 8-well LabTek chamber slides. When the PK-15 cells reached about 85% confluency, cells were transfected with the molecular DNA clone using Lipofectamine Plus Reagents according to the protocol supplied by the manufacturer (Life Technologies, Inc). Mock-transfected cells with empty pSK vector were included as controls. Three days after transfection, the cells were fixed with a solution containing 80% acetone and 20% methanol at 4° C. for 20 min., and an immunofluorescence assay using a PCV2-specific rabbit polyclonal antisera was performed to determine the in vitro infectivity of the molecular DNA clone.

To generate a biologically pure and homogeneous PCV2 infectious virus stock for the animal inoculation experiment, PK-15 cells free of PCV1 contamination were cultivated in T-25 culture flasks and transfected with the PCV2 molecular DNA clone. PK-15 cells were grown to about 85% confluency in T-25 flasks. The cells were washed once with sterile PBS buffer before transfection. For each transfection reaction in a T-25 flask, 12 μg of the PCV2 plasmid DNA was mixed with 16 μl of Plus Reagent in 0.35 ml of MEM media. A flask of mock-transfected cells with empty pSK vector was included as the negative control. After incubation at room temperature for 15 min., 50 μl of Lipofectamine Reagent diluted in 0.35 ml of MEM media was added to the mixture and incubated at room temperature for another 15 min. The transfection mixture was then added to a T-25 flask of PK-15 cells containing 2.5 ml of fresh MEM. After incubation at 37° C. for 3 hrs, the media was replaced with fresh MEM media containing 2% FBS and 1× antibiotics. The transfected cells were harvested at 3 days post transfection and stored at −80° C. until use. The infectious titer of the virus stock was determined by IFA.

Biologically pure and homogenous PCV2 infectious virus stock was generated by transfection of PK-15 cells with the PCV2 molecular DNA clone. PCV2 virions produced by in vitro transfection were infectious since the transfected cell lysates were successfully used to infect PK-15 cells. Thus, the PCV2 molecular DNA clone is capable of producing infectious PCV2 virions when transfected in vitro. The infectious titer of the homogenous PCV2 virus stock prepared from transfected cells was determined to be 1×10^{4.5} TCID₅₀/ml. This virus stock was used for inoculation of pigs. Lysates of cells mock-transfected with the empty pSK vector were unable to infect PK-15 cells.

### Example 3

### Virus Titration by Immunofluorescence Assay (IFA)

To determine the infectious titer of the homogenous PCV2 virus stock, PK-15 cells were cultivated on 8-well LabTek chamber slides. The virus stock was serially diluted 10-fold in MEM, and each dilution was inoculated onto 10 wells of the monolayers of the PK-15 cells growing on the LabTek chamber slides. Wells of non-inoculated cells were included as controls. The infected cells were fixed at 3 days post inoculation with a solution containing 80% acetone and 20% methanol at 4° C. for 20 min. After washing the cells with PBS buffer, the infected cells were incubated with a 1:1,000 diluted PCV2-specific rabbit polyclonal antibody (S. D. Sorden et al., “Development of a polyclonal-antibody-based fixed, paraffin-embedded tissue,” J. Vet. Diagn. Invest. 11:528-530 (1999)) at 37° C. for 1 hr. The cells were then washed three times with PBS buffer, and incubated with a secondary FITC-labeled goat anti-rabbit IgG (Kirkegaard & Perry Laboratories Inc, Gaithersburg, Md.) at 37° C. for 45 min. After washing the slides three times with PBS buffer, and the slides were mounted with fluoromount-G, cover-slipped and examined under a fluorescence microscope. The 50% tissue culture infectious dose per ml (TCID₅₀/ml) was calculated. Initially, cells were transfected with a plasmid construct containing a single copy of PCV2 genome but the infectious PCV2 titer from the single genome construct is much lower than the one containing the tandem genome. Therefore, the plasmid construct containing the dimeric form of PCV2 genome was used for the in vitro and in vivo transfection experiments.

### Example 4

### PCR-RFLP Analyses

The method for measuring PCV2 viremia is also described by Meng et al. (supra). To measure PCV2 viremia in pigs transfected with PCV2 molecular DNA clone and in pigs infected with PCV2 infectious virus stock, serum samples collected at different days post infection (DPI) were tested for the presence of PCV2 DNA by the general methods of a PCR-RFLP assay previously described (M. Fenaux et al., 2000, supra). Viral DNA was extracted from 50 μl of each serum sample using the DNAzol®. reagent according to the protocol supplied by the manufacturer (Molecular Research Center, Cincinnati, Ohio). The extracted DNA was resuspended in DNase-, RNase-, and proteinase-free water and tested for PCV2 DNA by PCR-RFLP (id.). PCR products from selected animals were sequenced to verify the origin of the virus infecting pigs.

Serum samples were collected from all control and inoculated animals at 0, 7, 14, 21, 28, and 35 DPIs and assayed for PCV2 viremia by detection of PCV2 DNA. The results show that PCV2 molecular DNA clone is infectious when injected directly into the liver and superficial iliac lymph nodes of SPF pigs. PCR products amplified from selected animals were sequenced. The sequence of the PCR products amplified from selected animals was identical to the corresponding region of the PCV2 molecular DNA clone.

### Example 5

### Construction of the Nonpathogenic PCV1 Infectious DNA Clone

The procedure used to construct a PCV1 infectious DNA clone is essentially the same as that described herein for PCV2 (See Meng et al., supra). Briefly, a pair of PCR primers, KPNPCV1.U set forth in SEQ ID NO: 21 and KPNPCV1.L set forth in SEQ ID NO: 22, was designed based on the published sequence of PCV1. This pair of primers amplifies the complete genome of PCV1 with an overlapping region containing the unique KpnI restriction enzyme site. The DNA of the PCV1 virus was extracted from the contaminated ATCC PK-15 cell line that was obtained from the American Type Culture Collection (ATCC accession number CCL-33). The PCV1 DNA was extracted from the ATCC PK-15 cells persistently infected with PCV1, using the QIAmp DNA minikit (Qiagen, Inc., Valencia, Calif.). The extracted DNA was amplified by PCR with AmpliTaq Gold Polymerase (Perkin-Elmer, Norwalk, Conn.). The PCR cycles consisted of an initial step of 95° C. for 10 min., followed by 35 cycles of denaturation at 94° C. for 1 min., annealing at 48° C. for 1 min., extension at 72° C. for 2 min., and a final extension at 72° C. for 7 min. The PCR product of expected size was separated by gel electrophoresis and purified by the glassmilk procedure using a Geneclean Kit (Bio 101, Inc., La Jolla, Calif.). The purified PCR product containing the complete PCV1 genome was first ligated into the advanTAge plasmid vector (Clontech, Palo Alto, Calif.). Escherichia coli DH5a competent cells were used for transformation. The recombinant plasmids were verified by restriction enzyme digestion. The full length PCV1 genomic DNA was excised from the advanTAge vector by digestion with KpnI restriction enzyme. The full-length PCV1 genomic DNA was ligated into pBluescript SK(+) (pSK) vector (Stratagene, La Jolla, Calif.) with T4 DNA ligase at 37° C. overnight. Recombinant plasmids containing the full-length PCV1 genome were isolated with a Qiagen plasmid mini kit (Qiagen, Valencia, Calif.) and were verified by restriction enzyme digestion and DNA sequencing. The full-length PCV1 genomic DNA was excised from the pSK vector by KpnI digestion, and dimerized to make the PCV1 infectious DNA clone as described above in Example 2 for the PCV2 infectious clone. These tandem dimers were made because the dimerized tandem DNA clones are advantageously found to be more efficient to transfect cells and produce infectious virions. To make the tandem dimer of the PCV1 DNA, the digested PCV1 genomic DNA was ligated with T4 DNA ligase at 37° C. for only 10 min., which favors the production of tandem dimers. The tandem dimers were subsequently cloned into pBluescript SK(+) (pSK) vector (Stratagene, La Jolla, Calif.). Recombinant plasmids containing tandem dimers of PCV1 genome (herein referred to as “PCV1 DNA clone”) were confirmed by PCR, restriction enzyme digestion, and DNA sequencing. The DNA concentration of the recombinant plasmids was determined spectrophotometrically.

The oligonucleotide primers employed were as follows:

Construction primers: PCV1 DNA clone construction KPNPCV1.U. Forward 5′-TTTGGTACCCGAAGGCCGATT-′3 (corresponds to SEQ ID NO:21); KPNPCV1.L. Backward 5′-ATTGGTACCTCCGTGGATTGTTCT-′3 (corresponds to SEQ ID NO:22); Hpa I-2 Backward 5′-GAAGTTAACCCTAAATGAATAAAAATAAAAACCATTACG-′3 PCV1-2 DNA clone construction (corresponds to SEQ ID NO:23); Nar I-3 Forward 5′-GGTGGCGCCTCCTTGGATACGTCATCCTATAAAAGTG-′3 PCV1-2 DNA clone construction (corresponds to SEQ ID NO:24); Psi I-5 Forward 5′-AGGTTATAAGTGGGGGGTCTTTAAGATTAA-′3 PCV1-2 DNA clone construction (corresponds to SEQ ID NO:25); Acl I-6 Backward 5′-GGAAACGTTACCGCAGAAGAAGACACC-′3 PCV1-2 DNA clone construction (corresponds to SEQ ID NO:26); Bgl-II-ORF2 Forward 5′-ACTATAGATCTTTATTCATTTAGAGGGTCTTTCAG-′3 PCV2-1 DNA clone construction (corresponds to SEQ ID NO:27); SpH-I-ORF2 Backward 5′-TACGGGCATGCATGACGTGGCCAAGGAGG-′3 PCV2-1 DNA clone construction (corresponds to SEQ ID NO:28); Bgl-II-PCV2 Backward 5′-AGACGAGATCTATGAATAATAAAAACCATTACGAAG-′3 PCV2-1 DNA clone construction (corresponds to SEQ ID NO:29); SpH-I-PCV2 Forward 5′-CGTAAGCATGCAGCTGAAAACGAAAGAAGTG-1-′3 PCV2-1 DNA clone construction (corresponds to SEQ ID NO:30).

Detection primers: MCV1 Forward 5′-GCTGAACTTTTGAAAGTGAGCGGG-′3 PCV1 and PCV2 detection (corresponds to SEQ ID NO:31); MCV2 Backward 5′-TCACACAGTCTCAGTAGATCATCCCA-′3 PCV1 and PCV2 detection (corresponds to SEQ ID NO:32); Orf.PCV1 Backward 5′-CCAACTTTGTAACCCCCTCCA-′3 PCV1 and PCV2-1 detection (corresponds to SEQ ID NO:33); Gen.PCV1 Forward 5′-GTGGACCCACCCTGTGCC-′3 PCV1 and PCV1-2 detection (corresponds to SEQ ID NO:34) Nested.Orf.PCV1 Backward 5′-CCAGCTGTGGCTCCATTTAA-′3 PCV1 and PCV2-1 detection (corresponds to SEQ ID NO:35); Nested.Gen.PCV1 Forward 5′-TTCCCATATAAAATAAATTACTGAGTCTT-′3 PCV1 and PCV1-2 detection (corresponds to SEQ ID NO:36); Orf.PCV2 Backward 5′-CAGTCAGAACGCCCTCCTG-′3 PCV2 and PCV1-2 detection (corresponds to SEQ ID NO:37); Gen.PCV2 Forward 5′-CCTAGAAACAAGTGGTGGGATG-′3 PCV2 and PCV2-1 detection (corresponds to SEQ ID NO:38); Nested.Orf.PCV2 Backward 5′-TTGTAACAAAGGCCACAGC-′3 PCV2 and PCV1-2 detection (corresponds to SEQ ID NO:39); Nested.Gen.PCV2 Forward 5′-GTGTGATCGATATCCATTGACTG-′3 PCV2 and PCV2-1 detection (corresponds to SEQ ID NO:40).

### Example 6

### Construction of a Chimeric PCV1-2 Viral DNA Clone

A chimeric virus was constructed between the nonpathogenic PCV1 and the PMWS-associated PCV2 by using infectious DNA clones of PCV1 and PCV2 (See Meng et al, supra). Briefly, to construct a chimeric PCV1-2 DNA clone, the ORF2 capsid gene of the nonpathogenic PCV1 was removed from the PCV1 infectious DNA clone, and replaced with the immunogenic ORF2 capsid gene of the pathogenic PCV2 in the genome backbone of PCV1. Two pairs of PCR primers were designed. The first primer pair for PCV2 ORF2, Psi I-5 set forth in SEQ ID NO: 25 and Acl 1-6 set forth in SEQ ID NO: 26, was designed with point mutations at the 5′ ends of the primers to create restriction enzyme sites AcII and PsiI to amplify the ORF2 gene of PCV2 and introduce flanking PsiI and AcII restriction enzyme sites by point mutation. The PCR reaction for the PCV2 ORF2 amplification consisted of an initial step at 95° C. for 9 min., followed by 38 cycles of denaturation at 95° C. for 1 min., annealing at 48° C. for 1 min., extension at 72° C. for 1 min., and a final extension at 72° C. for 7 min.

A second pair of PCR primers, Hpa I-2 set forth in SEQ ID NO: 23 and Nar I-3 set forth in SEQ ID NO: 24, was designed for the amplification of the pSK+ vector and its PCV1 genome insert. Point mutations were introduced at the 5′ ends of the PCR primers to create flanking restriction enzyme sites NarI and HpaI. This primer pair amplified the pSK+ vector and its insert PCV1 genomic DNA lacking the ORF2 capsid gene, that is, the PCV1 genome minus the PCV10RF2 (pSK-PCV1 δ ORF2) by using the PCV1 infectious DNA clone as the PCR template. The PCR reaction consisted of an initial step at 95° C. for 9 min., followed by 38 cycles of denaturation at 95° C. for 1 min., annealing at 50° C. for 1 min., extension at 72° C. for 3.5 min., and a final extension at 72° C. for 7 min. The PCV2 ORF2 PCR product was digested with the AcII and PsiI to remove the introduced point mutations. The pSK-PCV1 δ ORF2 product (the pSK vector-PCV1 genome PCR product lacking ORF2 gene of PCV1) was digested with the NarI and HpaI to remove the PCR introduced point mutations. The latter digestion produced a sticky end and a blunt end complementary to the PCV2 ORF2 PCR product digested by the AcII and PsiI restriction enzymes. The digested PCV2 ORF2 product and the ORF2-deleted pSK-PCV1 product were ligated with T4 DNA ligase to form the chimeric PCV1-2 genomic DNA clone, in which the ORF2 gene of PCV1 is replaced with the ORF2 gene of PCV2. Once the two PCR products were digested and religated, all the PCR introduced point mutations used to facilitate cloning were removed in the resulting chimeric clone. Escherichia coli DH5a competent cells were transformed. The recombinant plasmids containing the chimeric DNA clone were isolated and confirmed by PCR, restriction enzyme digestion and partial DNA sequencing. The full-length chimeric PCV1-2 genome was excised from the pSK+ vector (the recombinant plasmid) with kpnI digestion. The chimeric DNA genome was then dimimerized by a short 10-minute ligation reaction with T4 DNA ligase that favors the formation of linear dimers to produce the PCV 1-2 chimeric infectious DNA clone. The recombinant plasmids containing two copies of the chimeric viral genome were confirmed by PCR, restriction enzyme digestion and DNA sequencing.

### Example 7

### Evaluation of In Vitro Infectivity of PCV1-2 Chimeric DNA Clone

The viability of the chimeric PCV DNA clone (nonpathogenic PCV1 with the immunogenic capsid gene of PCV2) was tested in PK-15 cells as described in meng et al. (supra). When PK-15 cells were transfected with the chimeric viral DNA clone, viral antigen specific for PCV2 ORF2 capsid was detected by IFA at about 2 days post-transfection. The PCV1 capsid antigen was not detected in transfected cells. This experiment indicated that the chimeric DNA clone is infectious in vitro, is replicating in PK-15 cells and producing the immunogenic capsid protein of PCV2.

### Example 8

### Protection of Pigs Against PCV2 Infection Using a Chimeric PCV1-2 Vaccine

### Materials and Methods

### Vaccine Test Material

The vaccine was produced in Fort Dodge Animal Health (USA) and is referred to as Suvaxyn® PCV2 One Dose. This vaccine is an inactivated and adjuvanted vaccine for the stimulation of active immunity in pigs for protection against an infection with porcine circovirus type 2 (PCV2). Two milliliters (2 ml) of the vaccine is administered intramuscularly to pigs per dose. The active ingredient includes an inactivated chimeric porcine circovirus (cPCV)1-2, described by Meng et al (supra), having a relative potency of 1 (RP=1) and the adjuvant is a cyclodextrin derivative or a polyanionic polymer (as described in U.S. Pat. Nos. 6,165,995 and 6,610,310, respectively), Tween 80 and Squalane. The vaccine used in this study was batch number 2256-34-19 Apr. 2005 and the manufacturer was Fort Dodge Veterinaria, S.A. The DNA encoding the PCV1-2 chimeric circovirus used in the vaccine study described herein is shown in SEQ ID NO: 1.

### Challenge Strain

One of the PCV2 challenge strains used in the study was SN gg ING 8003 03DPF05. It was obtained from lymph node homogenates of specific pathogen free (SPF) pigs inoculated with PCV2 cDNA. These homogenates were used to inoculate SPF pigs; the lymph nodes of the pigs were homogenized and titrated, to be used as challenge virus. The GenBAnk accession number for the U.S. type 2A challenge strain is AF264042 (SEQ ID NO: 9). The GenBAnk accession number for the European type 2B challenge strain is AJ623306 (SEQ ID NO: 11). The capsid protein of the U.S. type 2 A challenge strain is found in SEQ ID NO: 10, and the capsid protein of the European type 2B challenge strain is found in SEQ ID NO: 12.

Before challenge and after challenge of the pigs in the present study, titrations of the inoculum were performed on SK cells. Pigs were inoculated with a dose of 10⁵⁵ TCID₅₀/pig (6 ml/pig at 1048 TCID₅₀/ml).

### Test Animals

The study was carried out in 86 three to four week old (from 19 days-old to 31 days-old) conventional pigs, serologically negative or with low antibody titers to PCV2 These pigs were obtained from the Mas El Cros farm (Spain).

The pigs were given water and food ad libitum throughout the experiment. The feed was Porquina Sprint form Carhill Spain batch number 74871.

### Facilities

The in vivo experiment was carried out in the challenge facilities of Fort Dodge Veterinaria S.A. During the vaccination period, pigs of groups #1, #2 and #3 were housed in the Cal Menut farm (Ripoll, Spain) between dates 1 Sep. 2006 and 8 Nov. 2006 (the day before the challenge). Pigs of group #4, were housed in the Cal Menut farm from 1 Sep. 2006 until 29 Nov. 2006 (the day of slaughter). The laboratory work was performed at the R&D laboratory at Fort Dodge Veterinaria S.A.

### Experimental Design

### Treatment Groups

86 three to four-week-old pigs from sows of the Mas El Cros farm, seronegative or with low antibody titers to PCV2, were selected and divided into 4 groups as follows:

1: One-shot group (22 pigs): vaccinated once, challenged

2: Two-shots group (22 pigs): vaccinated twice, challenged

3: Control group (22 pigs): non-vaccinated, challenged

4: Control group (20 pigs): non-vaccinated, non-challenged

TABLE 1
DESCRIPTION OF TREATMENT GROUPS

| | 1^{st} vaccination | 2^{nd} vaccination | Challenge |
|---|---|---|---|
| Group | (3-4 weeks old) | (6-7 weeks old) | (20-21 weeks old) |
| 1 | yes | No | yes |
| 2 | yes | Yes | yes |
| 3 | no | No | yes |
| 4 | no | No | no |

The pigs were divided into four groups according to the following criteria: Antibody titers against PCV2 (IPMA) at reception; age of pigs; and genus. The objective was to make the four groups as similar as possible.

Parameters Evaluated Pre-Challenge (D=day):

Serology [symbol] D-1, D18, D35, D68, D102, D132 PV (D0 PI)

### Parameters Evaluated Post-Challenge:

Rectal temperature [symbol] D0, D2, D5, D7, D9, D12, D14, D16, D20, D21 PI

Bodyweight [symbol] D0, D7, D14, D21PI

Serology [symbol] D0, D7, D14, D21 PI

Viremia [symbol] D0, D7, D14, D21 PI

Histopathology [symbol] D21 PI

The rectal temperatures post-infection (PI) and body weights PI of pigs belonging to group 4 were not considered for the analysis of the results as this group was not challenged (group 4). The usefulness of this group of pigs was as a control for the histopathological lesions, serology and viremia.

### Vaccination Protocol

Pigs of group 1 were vaccinated with one dose (2 ml) at 3-4 weeks of age; pigs of group 2 were vaccinated with one dose (2 ml) at 3-4 weeks of age, and revaccinated 3 weeks later, at 6-7 weeks of age.

Each dose of 2 ml was administered by deep intramuscular route, in the neck, close to the ear (right side for vaccination and left side for revaccination), using a sterile disposable 2 ml syringe fitted with 1.1 mm×40 mm needle.

Control pigs (groups 3 and 4) were left unvaccinated.

### Challenge Protocol

Pigs were challenged 19 weeks after vaccination (group 1), or 16 weeks after the 2^{nd} vaccination (group 2). All pigs, including group 3 pigs, were around 20-21 weeks of age at the time of challenge. Control pigs (group 4) were left unchallenged.

Pigs were inoculated with the challenge strain of PCV2. Pigs received 4 ml by intranasal (IN) route, and 2 ml by intramuscular (IM) route. The IN inoculation were done using 5 ml syringes and the IM inoculation using 2 ml syringes and 1.1×40 mm needles. The IN route was chosen since it is the natural route of infection, and the IM route to enhance the probabilities of infection.

### Rectal Temperatures PI

Rectal temperatures were recorded the days indicated above.

### Body Weights PI

The body weight of the pigs was recorded at D0, D7, D14 and D21PI, using the scales Santaularia (1-300 kg).

### Serology

Blood samples were collected at D-1, D18, D35, D68, D102 and D132 post vaccination (PV), (D0 PI), and at D0, D7, D14, and D21 PI in tubes for obtaining serum. These samples were tested for the presence of antibodies against PCV2, using the PCV2 IPMA technique, and by ELISA test

The ELISA test procedure consisted of a modified indirect ELISA based on recombinant baculovirus-expressed PCV2 capsid protein (Nawagitgul, P., Harms, P. A., Morozov, I., Thacker, B. J., Sorden, S. D., Lekcharoensuk, C., and Paul, P. S.

Modified indirect porcine circovirus (PCV) type 2-based and recombinant capsid protein (ORF2)-based enzymed-linked immunosorbent assays for detection of antibodies to PCV. Clin. Diagn. Lab. Immunol.; 9: 33-40, 2002). Briefly, the PCV2 antigen-coated plate was washed three times using PBST washing buffer (0.1 M PBS-pH7.2 and 1% Tween 20). Sera were diluted 1:6000 in 5% milk diluent, and 100 μL of each diluted serum was incubated with positive and negative antigen at 36±2° C. for 1 h. Excess antibodies were removed by washing 3 times with PBST buffer. Then, 100 μL of diluted peroxidase-labeled anti-pig IgG was added to each well, and incubated at 36±2° C. for 1 h. After washing 3 times to remove excess secondary antibody, 100 μL of 3,3′, 5, 5′ tetramethylbenzidine (TMB) substrate was added and incubated for 20 min at 36±2° C. The reaction was not stopped for reading. The optical density value was measured at 650 nm minus 420 nm using a microplate reader, and reported as the sample/positive control (S/P) ratio.

S/P ratio=OD sample−OD negative control/OD positive control−OD negative control

Sera with S/P ratios≧0.5 were considered positive.

### Viremia

Serum samples taken at D-1PV, D18 PV and at D0, D7, D14, and D21 PI were used for measuring viremia,

DNA purification from serum samples was performed using standard protocols known to those skilled in the art.

For the quantification of PCV2 viremia, a real-time PCR technique adapted from a previously published method was performed. (Olvera, A., Sibila, M., Calsamiglia, M., Segales, J., Domingo, M. Comparison of porcine circovirus type 2 load in serum quantified by a real time PCR in postweaning multisystemic wasting sindrome and porcine dermatitis and nephropathy sindrome naturally affected pigs. J. Virol. Meth.; 117: 75-80, 2004).

PCV2-specific PCR testing was used to detect the presence of PCV2 viral genomic DNA in serum samples. Viral genomic DNA was purified following standard procedures known to those skilled in the art. PCV2 specific sequences were measured using PCR, following standard procedures known to those skilled in the art. A 592-bp fragment was amplified by using ABI AmpliTaq Gold DNA polymerase and gene-specific primers: F1PCV2,5′-ATGCCCAGCMGAAGAATGG-3′ (SEQ ID NO: 41) and RPCV2,5′-TGGTTTCCAGTATGTGGTTTCC-3′ (SEQ ID NO: 42). The purified viral DNA was used as template and denatured at 95° C. for 10 min. The PCR program of reactions consisted of 35 cycles of denaturation at 94° C. for 30 sec, annealing at 59° C. for 1 min, and extension at 72° C. for 1 min. Ten μL of PCR product were used to detect 592 bp PCV2 DNA fragment by agarose gel electrophoresis.

### Gross Pathology, Histopathology and In Situ Hybridization

At D21 PI all pigs were euthanized and necropsied. Gross lesions were recorded. Tissue samples (inguinal superficial lymph node, tracheobronchial lymph node, submandibular lymph node, lung, tonsil, spleen, liver and kidney) were obtained and placed in 10% buffered formalin to perform histopathology and in situ hybridization (ISH).

Histopathology: tissue portions of 2-3 mm were allocated in plastic cassettes, and dehydrated in graded alcohols and paraffin-embedded using an automatic tissue processor system. Tissue blocks were done, and 4-5 μm sections cut using an automatic microtome. Sections were stained with haematoxilin-eosin using an automatic stainer and evaluated with an optic microscope.

### In Situ Hybridization

In situ hybridization was performed and tissue sections were evaluated microscopically. (Kennedy, S., Segales, J., Rovira, A., Scholes, S., Domingo, M., Moffet, D., Meehan, B., O'Neill, R., McNeilly, F., Allan, G. Absence of evidence of porcine circovirus infection in piglets with congenital tremors. J. Vet. Diagn. Invest.; 15(2): 151-156, 2003)

Microscopic lesions were scored according to the published classification of Chianini et al. (Chianini, F., Majó, N., Segalés, J., Domínguez, J., Domingo, M. Immunohistochemical characterisation of PCV2 associate lesions in lymphoid and non-lymphoid tissues of pigs with natural postweaning multisystemic wasting syndrome (PMWS)). The lesions in each tissue were scored, and a final score, as described below, was emitted for each animal.

Stage 0: no microscopic lesions observed

Stage 1: In lymphoid tissues, mild lymphocyte depletion and mild infiltration of histiocytes and a few multinucleated giant cells, mainly in the germinal centers of follicular areas. In some cases, mild interstitial pneumonia, nephritis and/or hepatitis.

Stage 2: In lymphoid tissues, moderate lymphocyte depletion and moderate infiltration of histiocytes and multinucleated giant cells, mainly in follicular and interfollicular areas. In some cases, mild interstitial pneumonia, nephritis and/or hepatitis.

Stage 3: In lymphoid tissues, severe lymphocyte depletion and severe infiltration of histiocytes and multinucleated giant cells, in drastically reduced follicules, interfollicular and medulla-like areas. In some cases, presence of cytoplasmic basophilic inclusions in histiocytes. In some cases, moderate to severe interstitial pneumonia, nephritis and/or hepatitis.

PCV2 nucleic acid detection was scored according to the published classification of Chianini et al. (Chianini, F., Majó, N., Segalés, J., Domínguez, J., Domingo, M. Immunohistochemical characterisation of PCV2 associate lesions in lymphoid and non-lymphoid tissues of pigs with natural postweaning multisystemic wasting syndrome (PMWS) Vet. Immunol. Immunopathol.; 94(1-2):63-75, 2003). The amount of PCV2 nucleic acid in each tissue was scored, and a final score was emitted for each animal.

Stage 0: no PCV2 nucleic acid detected

Stage 1: PCV2 nucleic acid confined in infiltrating macrophages and dendritic cells in follicular areas in lymphoid tissues.

Stage 2: PCV2 nucleic acid detection in infiltrating macrophages, multinucleated giant cells and dendritic cells of the cortex of lymph nodes. In tonsil, detection in macrophages and dendritic cells of follicular areas. In some cases, PCV2 detected in histiocytic cells of PALS, in the spleen. In some cases, PCV2 detected in histiocytic cells of BALT, in lung. In some cases, PCV2-positive Kupffer cells occasionally observed in liver. In some cases, PCV2 antigen detection in lymphoplasmacytic infiltration in kidney.

Stage 3: PCV2 distribution in lymphoid tissues similar to stage 2, but nucleic acid also detected in macrophages of the medulla-like area of lymph nodes. In some cases, antigen detected in alveolar septae and peribronchial/bronchiolar macrophages in lung. In some cases, detection in Kupffer cells and perilobular and periportal macrophages in liver.

### Results

### Rectal Temperatures PI

No statistically significant differences regarding rectal temperatures (RT) PI were observed between vaccinated (1-shot/2-shots) and controls.

In the 1-shot vaccinated and challenged group, the maximum rectal temperature achieved by individual pigs was 40.7° C.; in the 2-shots vaccinated and challenged group, the maximum rectal temperature achieved by individual pigs was 40.8° C.; while in the non-vaccinated and challenged group the highest rectal temperature achieved was 40.9° C.

There were no statistically significant differences (one-tailed t-Test with 5% of significance level) PI between the groups regarding rectal temperatures PI, except between groups 2 and 3 at day 2 PI (the temperatures of group 2 were also higher at D0, the day of challenge), which was probably influenced by the handling of the pigs and not because of the PCV2 virus, as it was too close to the challenge.

### Body Weights

The relative mean daily gain was calculated and there were no statistically significant differences between vaccinated (1-shot/2-shots) and non-vaccinated and challenged group, even the Relative Mean Daily Gain (RMDG) of the controls was lower than the ones of the 1-shot and 2-shots vaccinated pigs, as shown in FIG. 3. The non-vaccinated and challenged pigs (group 3) gained (per day) a mean of 133 g less than the 1-shot vaccinated pigs; and a mean of 95.5 g less that the 2-shot vaccinated pigs.

### Serology

### Serology Post-Vaccination: Antibody Titers Tested by IPMA and ELISA

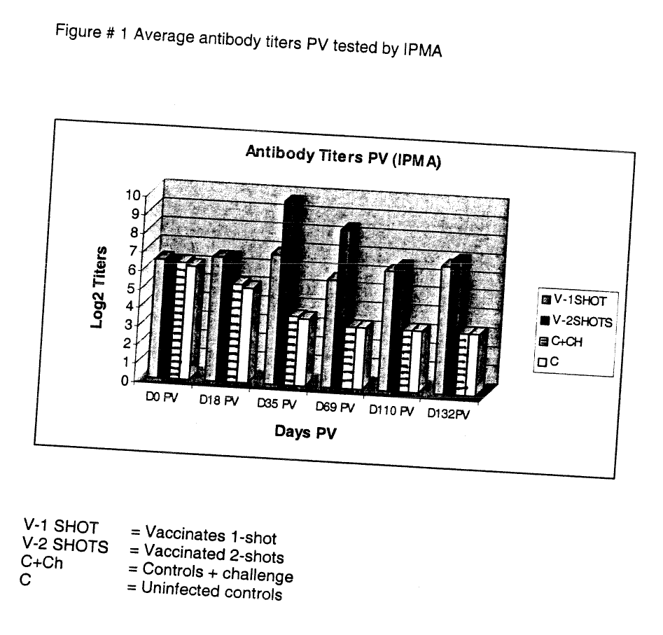

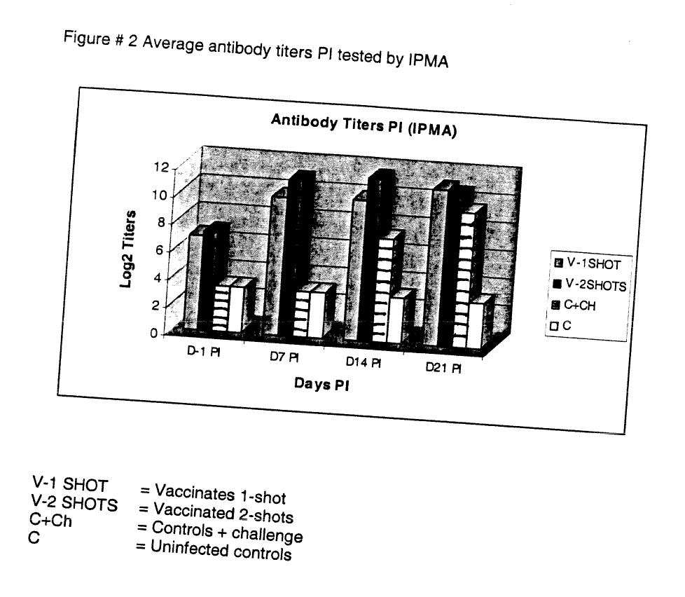

The antibody titers of pigs vaccinated with 1-shot (Group 1), 2-shots (Group 2), and the Control pigs of Groups 3 and 4 are shown below in Tables 2-5 and in FIGS. 1 and 2. (See Table 1 for group designations.)

### Average Antibody Titers PV Tested by IPMA

TABLE 2
GEOMETRIC MEANS OF ANTIBODIES PV (IPMA)

| GROUP | D0 PV | D18 PV | D35 PV | D69 PV | D110 PV | D132PV |
|---|---|---|---|---|---|---|
| 1 | 80.0 | 95.1 | 125.5 | 56.6 | 80.0 | 96.0 |
| 2 | 85.7 | 133.3 | 921.8 | 361.6 | 102.2 | 145.9 |
| 3 | 77.5 | 38.8 | 13.0 | 10.0 | 10.0 | 10.0 |
| 4 | 67.3 | 34.6 | 12.4 | 10.0 | 10.0 | 10.0 |

D0 PV = Day of 1^{st} vaccination
D21 PV = Day of 2^{nd} vaccination
D132 PV = Day of challenge

### Percentage of Positive Animals PV Tested by ELISA

TABLE 3
ELISA PERCENTAGE OF POSITIVES PV

| GROUP | D0 PV | D18 PV | D35 PV | D69 PV | D110 PV | D132 PV |
|---|---|---|---|---|---|---|
| 1 | 55% | 25% | 80% | 85% | 95% | 78.9% |
| 2 | 35% | 21% | 89.5% | 94.1% | 94.1% | 100% |
| 3 | 45.4% | 31.8% | 14.3% | 4.7% | 4.7% | 5% |
| 4 | 47.3% | 21% | 10.5% | 0% | 0% | 6.2% |

D0 PV = Day of 1^{st} vaccination
D21 PV = Day of 2^{nd} vaccination
D132 PV = Day of challenge

### Average Antibody Titers PI Tested by IPMA

TABLE 4
GEOMETRIC MEANS OF ANTIBODIES PI (IPMA)

| GROUP | D-1 PI | D7 PI | D14 PI | D21 PI |
|---|---|---|---|---|
| 1 | 96.0 | 1002.2 | 1043.9 | 2562.8 |
| 2 | 145.9 | 2444.4 | 3537.7 | 1998.6 |
| 3 | 10.0 | 10.0 | 176.7 | 874.3 |
| 4 | 10.0 | 10.0 | 17.9 | 11.3 |

### Percentage of Positive Animals PV Tested by ELISA

TABLE 5
5 ELISA PERCENTAGE OF POSITIVES PI

| GROUP | D-1 PI | D7 PI | D14 PI | D21 PI |
|---|---|---|---|---|
| 1 | 78.9% | 100% | 94.4% | 93.7% |
| 2 | 100% | 100% | 100% | 100% |
| 3 | 5% | 5% | 23.8% | 15% |
| 4 | 6.2% | 6.2% | 0% | 0% |

The differences between the IPMA titers between the 1-shot and 2-shot groups post vaccination were statistically significant at D35PV and D69PV, but not at days D110PV and D132 PV. Differences between vaccinates (1-shot/2-shots) and controls (uninfected controls/controls+challenge) were statistically significant from D18 PV to D132 PV.

Post-infection, the differences concerning the IPMA titers between vaccinates 1-shot and 2-shot were only statistically significant at D7PI. Differences between vaccinates (1-shot/2-shots) and controls (uninfected controls/controls+challenge) were statistically significant at D-1 PI, D7 PI and D14 PI.

### Viremia

### Real-Time PCR

The results of PCV2 real-time PCR are expressed in the following tables; results are expressed as PCV2 genome copy numbers per ml of serum.

TABLE 6
GROUP 1 (PIGS VACCINATED WITH 1-SHOT + CHALLENGE)

| | Pig # | | | | | |
|---|---|---|---|---|---|---|
| | D0 PV | D18 PV | D0 PI | D7 PI | D14 PI | D21 PI |
| Average | 0 | 0 | 0 | 0 | 0 | 0 |
| % positive pigs | 0 | 0 | 0 | 0 | 0 | 0 |

PV: post-vaccination;
PI: post-infection;
ND: not done

TABLE 7
GROUP 2 (PIGS VACCINATED WITH 2-SHOT + CHALLENGE)

| | Pig # | | | | | |
|---|---|---|---|---|---|---|
| | D0 PV | D18 PV | D0 PI | D7 PI | D14 PI | D21 PI |
| Average | 0 | 0 | 0 | 0 | 14.40 | 0 |
| % positive pigs | 0 | 0 | 0 | 0 | 6.66 | 0 |

PV: post-vaccination;
PI: post-infection;
ND: not done

TABLE 8
GROUP 3 (CONTROL PIGS + CHALLENGE)

| | Pig # | | | | | |
|---|---|---|---|---|---|---|
| | D0 | D18 | D0 PI | D7 PI | D14 PI | D21 PI |
| Average | 0 | 0 | 0 | 89606.19 | 32732.14 | 4252.86 |
| % positive pigs | 0 | 0 | 0 | 85.71 | 80.95 | 76.19 |

PI: post-infection;
ND: not done

TABLE 9
GROUP 4 (CONTROL PIGS)

Pig #

| | D0 | D18 | D0 | D7 | D14 | D21 |
|---|---|---|---|---|---|---|
| Average | 0 | 0 | 0 | 0 | 0 | 0 |
| % positive pigs | 0 | 0 | 0 | 0 | 0 | 0 |

ND: not done

PCV2 genome was not detected at D0 PV in any pig in the experiment. All the pigs remained non-viremic throughout the postvaccinal period.

During the postinoculation period, no virus was detected in the serum of Group 4 controls.

In group 3 (control+challenge), PCV2 genome was detected after challenge in all but one pig. The peak of viremia was detected at D7PI, with a mean of 89606.19 PCV2 genome copy numbers/ml.

In group 1 (vaccinated 1-shot+challenge), no virus was detected in the serum after challenge.

In group 2 (vaccinated 2-shot+challenge), no virus was detected in the serum after challenge, except for one pig (216 PCV2 genome copy numbers/ml at D14 PI).

Statistically significant differences (p≦0.05) were observed as follows:
at D7 PI:
   between uninfected Controls and Controls+Challenge
   between Controls+challenge and Vaccinated 1-shot
   between Controls+challenge and Vaccinated 2-shots
at D14 PI:
   between uninfected Controls and Controls+Challenge
   between Controls+Challenge and Vaccinated 1-shot
   between Controls+Challenge and Vaccinated 2-shots
at D21 PI:
   between Controls and Controls+Challenge
   between Controls+Challenge and Vaccinated 1-shot
   between Controls+Challenge and Vaccinated 2-shots

### Gross Lesions

Gross lesions were present in all groups, but they were very mild and affected very few pigs.

In control pigs (group 4), only one animal presented an enlargement of one kidney and dilatation of the medulla, due to ureter obstruction.

In non-vaccinated and challenged pigs (group 3), the main lesions observed were lymphadenopathy (lymph nodes increased in size) of particular lymph nodes or generalized; areas of cranioventral consolidation in lung; and white-spotted kidneys.

Very similar lesions were observed in vaccinated and challenged pigs (groups 1 and 2).

The results of gross lesions scoring are expressed in the following tables 10-13.

TABLE 9
GROUP 1 (PIGS VACCINATED WITH 1-SHOT + CHALLENGE)

| | Lymph nodes | | | | | | | | |
|---|---|---|---|---|---|---|---|---|---|
| | | Tracheo- | Inguinal | | | | | | |
| Pig | Submandibular | bronchial | superficial | Tonsil | Lung | Spleen | Liver | Kidney | SCORE |
| Average | 0 | 0 | 0 | 0 | 0.06 | 0 | 0 | 0.06 | 0.13 |

TABLE 10
GROUP 2 (PIGS VACCINATED WITH 2-SHOT + CHALLENGE)

| | Lymph nodes | | | | | | | | |
|---|---|---|---|---|---|---|---|---|---|
| | | Tracheo- | Inguinal | | | | | | |
| Pig | Submandibular | bronchial | superficial | Tonsil | Lung | Spleen | Liver | Kidney | SCORE |
| Average | 0.07 | 0 | 0 | 0 | 0.21 | 0 | 0 | 0.14 | 0.43 |

TABLE 11
GROUP 3 (CONTROL PIGS + CHALLENGE)

| | Lymph nodes | | | | | | | | |
|---|---|---|---|---|---|---|---|---|---|
| | | Tracheo- | Inguinal | | | | | | |
| Pig | Submandibular | bronchial | superficial | Tonsil | Lung | Spleen | Liver | Kidney | SCORE |
| Average | 0.10 | 0.05 | 0.10 | 0 | 0.10 | 0 | 0 | 0 | 0.33 |

TABLE 12
GROUP 4 (CONTROL PIGS)

| | Lymph nodes | | | | | | | | |
|---|---|---|---|---|---|---|---|---|---|
| | | Tracheo- | Inguinal | | | | | | |
| Pig | Submandibular | bronchial | superficial | Tonsil | Lung | Spleen | Liver | Kidney | SCORE |
| Average | 0 | 0 | 0 | 0 | 0.05 | 0 | 0 | 0.05 | 0.11 |

### Histopathology

The results of the histopathology scoring are expressed in the following tables 14-17.

TABLE 13
GROUP 1 (PIGS VACCINATED WITH 1-SHOT + CHALLENGE)

| | | Lymph nodes | | Tonsil | | Spleen | | Liver | Kidney | Lung |
|---|---|---|---|---|---|---|---|---|---|---|
| Pig # | Stage* | Depletion | Infiltration | Depletion | Infiltration | Depletion | Infiltration | Hepatitis | Nephritis | Pneumonia |
| Average | 0.06 | 0.06 | 0.06 | 0 | 0 | 0 | 0 | 0.18 | 0.18 | 0.06 |

TABLE 14
GROUP 2 (PIGS VACCINATED WITH 2-SHOT + CHALLENGE)

| | | Lymph nodes | | Tonsil | | Spleen | | Liver | Kidney | Lung |
|---|---|---|---|---|---|---|---|---|---|---|
| Pig # | Stage* | Depletion | Infiltration | Depletion | Infiltration | Depletion | Infiltration | Hepatitis | Nephritis | Pneumonia |
| Average | 0.07 | 0 | 0.07 | 0 | 0 | 0 | 0 | 0.21 | 0.07 | 0.36 |

TABLE 15
GROUP 3 (CONTROL PIGS + CHALLENGE)

| | | Lymph nodes | | Tonsil | | Spleen | | Liver | Kidney | Lung |
|---|---|---|---|---|---|---|---|---|---|---|
| Pig # | Stage* | Depletion | Infiltration | Depletion | Infiltration | Depletion | Infiltration | Hepatitis | Nephritis | Pneumonia |
| Average | 0.38 | 0.19 | 0.48 | 0 | 0.05 | 0.05 | 0.10 | 0.10 | 0 | 0.24 |

TABLE 16
GROUP 4 (CONTROL PIGS)

| | | Lymph nodes | | Tonsil | | Spleen | | Liver | Kidney | Lung |
|---|---|---|---|---|---|---|---|---|---|---|
| Pig # | Stage* | Depletion | Infiltration | Depletion | Infiltration | Depletion | Infiltration | Hepatitis | Nephritis | Pneumonia |
| Average | 0 | 0 | 0 | 0 | 0 | 0 | 0 | 0 | 0 | 0 |

Statistically significant differences (p≦0.05) were observed as follows:

Lymph node depletion:
between uninfected Controls and Controls+Challenge;
between Controls+challenge and Vaccinated 2-shots

Lymph node infiltration:
between uninfected Controls and Controls+Challenge;
between Controls+challenge and Vaccinated 1-shot
between Controls+challenge and Vaccinated 2-shots

Hepatitis:
between uninfected controls and Vaccinated 2-shots

Nephritis:
between uninfected controls and Vaccinated 1-shot;
between Controls+Challenge and Vaccinated 1-shot

Pneumonia:
between uninfected controls and Vaccinated 2-shots

Stage:
between uninfected Controls and Controls+Challenge;
Controls+challenge and Vaccinated 1-shot;
Controls+challenge and Vaccinated 2-shots

The percentage of animals with microscopic lesions in each group is expressed in the following table.

TABLE 17
PERCENTAGE (%) OF PIGS WITH MICROSCOPIC LESIONS

| | Lymph | | | | | |
|---|---|---|---|---|---|---|
| Group | nodes | Tonsil | Spleen | Liver | Kidney | Lung |
| 1 | 5.88 | 0 | 0 | 17.64 | 17.64 | 5.88 |
| vaccinated 1- | | | | | | |
| shot + challenge | | | | | | |
| 2 | 7.14 | 0 | 0 | 21.42 | 7.14 | 21.42 |
| vaccinated 2- | | | | | | |
| shot + challenge | | | | | | |
| 3 | 38.09 | 4.76 | 9.52 | 4.76 | 0 | 23.80 |
| control + | | | | | | |
| challenge | | | | | | |
| 4 | 0 | 0 | 0 | 0 | 0 | 0 |
| control | | | | | | |

Pigs of group 2 showed typical mild lesions of PCV2 infection (stage 1). In contrast, pigs of group 1 showed very similar lesions, but in a lower percentage.

### In Situ Hybridization

The results of in situ hybridization scoring are expressed in the following tables.

TABLE 18
GROUP 1 (PIGS VACCINATED WITH 1-SHOT + CHALLENGE)

| | | Lymph | | | | | |
|---|---|---|---|---|---|---|---|
| Pig | Stage* | nodes | Tonsil | Spleen | Liver | Kidney | Lung |
| Average | 0 | 0 | 0 | 0 | 0 | 0 | 0 |

Stage of disease is according to Chianini et al. (Chianini, F., Majó, N., Segalés, J., Domínguez, J., Domingo, M. Immunohistochemical characterisation of PCV2 associate lesions in lymphoid and non-lymphoid tissues of pigs with natural postweaning multisystemic wasting syndrome (PMWS). Vet. Immunol. Immunopathol.; 94(1-2):63-75, 2003.)

TABLE 19
GROUP 2 (PIGS VACCINATED WITH 2-SHOT + CHALLENGE)

| | | Lymph | | | | | |
|---|---|---|---|---|---|---|---|
| Pig | Stage* | nodes | Tonsil | Spleen | Liver | Kidney | Lung |
| Average | 0 | 0 | 0 | 0 | 0 | 0 | 0 |

TABLE 20
GROUP 3 (CONTROL PIGS + CHALLENGE)

| | | Lymph | | | | | |
|---|---|---|---|---|---|---|---|
| Pig | Stage* | nodes | Tonsil | Spleen | Liver | Kidney | Lung |
| Average | 0.33 | 0.29 | 0.24 | 0.10 | 0 | 0 | 0 |

Stage of disease is according to Chianini et al. (Chianini, F., Majó, N., Segalés, J., Domínguez, J., Domingo, M. Immunohistochemical characterisation of PCV2 associate lesions in lymphoid and non-lymphoid tissues of pigs with natural postweaning multisystemic wasting syndrome (PMWS). Vet. Immunol. Immunopathol.; 94(1-2):63-75, 2003.)

TABLE 21
GROUP 4 (CONTROL PIGS)

| | | Lymph | | | | | |
|---|---|---|---|---|---|---|---|
| Pig | Stage* | nodes | Tonsil | Spleen | Liver | Kidney | Lung |
| Average | 0 | 0 | 0 | 0 | 0 | 0 | 0 |

Stage of disease is according to Chianini et al. (Chianini, F., Majó, N., Segalés, J., Domínguez, J., Domingo, M. Immunohistochemical characterisation of PCV2 associate lesions in lymphoid and non-lymphoid tissues of pigs with natural postweaning multisystemic wasting syndrome (PMWS). Vet. Immunol. Immunopathol.; 94(1-2):63-75, 2003.)

Statistically significant differences (p≦0.05) were observed as follows:

Lymph nodes:

between uninfected Controls and Controls+Challenge

between Controls+challenge and Vaccinated 1-shot

between Controls+challenge and Vaccinated 2-shots
Tonsil:

between uninfected Controls and Controls+Challenge

between Controls+challenge and Vaccinated 1-shot

between Controls+challenge and Vaccinated 2-shots
Stage:

between uninfected Controls and Controls+Challenge

between Controls+challenge and Vaccinated 1-shot

between Controls+challenge and Vaccinated 2-shots

The percentage of animals with PCV2 nucleic acid detected in each group are expressed in the following table.

TABLE 22
PERCENTAGE (%) OF PIGS WITH PCV2 NUCLEIC
ACID IN TISSUES

| | Lymph | | | | | |
|---|---|---|---|---|---|---|
| Group | nodes | Tonsil | Spleen | Liver | Kidney | Lung |
| 1 | 0 | 0 | 0 | 0 | 0 | 0 |
| vaccinated 1- | | | | | | |
| shot + challenge | | | | | | |
| 2 | 0 | 0 | 0 | 0 | 0 | 0 |
| vaccinated 2- | | | | | | |
| shot + challenge | | | | | | |
| 3 | 28.57 | 23.80 | 9.52 | 0 | 0 | 0 |
| control + | | | | | | |
| challenge | | | | | | |
| 4 | 0 | 0 | 0 | 0 | 0 | 0 |
| control | | | | | | |

PCV2 nucleic acid was only detected in tissues of non-vaccinated and challenged pigs (group 3). The amount of nucleic acid detected in all cases was very low.

### Discussion

The construction of an infectious DNA clone based on the capsid protein of PCV2 and the backbone of PCV1 had previously been described and characterized (Fenaux, M., Opriessnig, T., Halbur, P. G., Meng, X. J. Immunogenicity and pathogenicity of chimeric infectious DNA clones of pathogenic porcine circovirus type 2 (PCV2) and nonpathogenic PCV1 in weanling pigs. J. Virol.; 77(20):11232-43, 2003).

The resulting virus, called chimeric PCV1-2 (cPCV1-2), was demonstrated to be attenuated for pigs but also immunogenic in front of the challenge with PCV2 (Fenaux, M., Opriessnig, T., Halbur, P. G., Elvinger, F., Meng, X. J. A chimeric porcine circovirus (PCV) with the immunogenic gene of the pathogenic PCV type 2 (PCV2) cloned into the genomic backbone of the non-pathogenic PCV1 induces protective immunity against PCV2 infection in pigs. J. Virol.; 78(12): 6297-6303, 2004). The donor virus of the capsid protein of PCV2 was a North American PCV2A strain, as described previously.

In the study presented herein, it has been demonstrated that the immunity induced by this vaccine, administered in a 1-shot or in a 2-shot immunization scheme, is able to reduce and/or prevent the pathogenic effects of the subsequent challenge of pigs with a wild type PCV2B of European origin, when the challenge is done 4 months after vaccination.

The rectal temperatures of vaccinated and challenged pigs (groups 1 and 2), and those of non-vaccinated and challenged pigs (group 3) were not statistically different in any of the days of measurement. The exception was between groups 2 and 3 at day 2 PI (the temperatures of group 2 were also higher at D0, the day of challenge), which was probably influenced by the handling of the pigs and not because of the PCV2 virus, as it was too close to the challenge.

Also, neither the mean body weights nor the relative weight gain (RMDG) was statistically different. Furthermore, the RMDG of the controls was lower than that of the 1-shot and 2-shots vaccinated pigs. The non-vaccinated and challenged pigs (group 3) gained a mean of 133 g less than the 1-shot vaccinated pigs per day; and a mean of 95.5 g less than that of the 2 shot vaccinated pigs.

Consequently, it was not possible to measure any potential differences between vaccinated and non-vaccinated pigs, as regards to the above-noted clinical parameters.

The four groups of pigs had maternal antibodies at the time of vaccination (detected by IPMA and ELISA).

In pigs administered 1-shot of the vaccine followed by challenge (group 1), the highest titers were observed at 35 days after the vaccination, declining until challenge. 68.4% (13/19) of the animals were positive as shown by IPMA. 78.9% (15/19) of the animals were positive as shown by ELISA at challenge. At 7 day PI, a strong anamnestic response to PCV2 was observed in all the pigs by IPMA (with titers ranging from 1:80 to 1:5120) and all of the pigs were positive. At D14 PI and D21 PI IPMA, antibody titers ranged between 320 and 20480. 77.7% of the animals were positive at 14D PI and 100% at D21 PI by IPMA.

In pigs administered 2-shots of the vaccine followed by challenge (group 2), strong seroconversion was observed after the booster (D35 PV), declining until challenge. 66.6% (10/15) of the animals were positive as shown by IPMA). At 7 days PI, a strong anamnestic response to PCV2 was observed in all the pigs by IPMA (with titers ranging from 1:1280 to 1:20480). At D14 PI IPMA, antibody titers ranged between 320 and 20480.

In the control groups, the maternally derived antibody levels declined at D18 and were undetectable at D69 by IPMA. After challenge, control pigs (group 3) seroconverted slowly. The unchallenged control pigs remained seronegative until necropsy.

The main drawback of the real-time PCR was that it was not able to differentiate between the genome of the vaccinal virus and the genome of the wild type virus. However, the real-time PCR from sera obtained at D18 PV yielded negative results in all pigs tested. Then, it was assumed that the positive real-time PCR results obtained after challenge were always due to viremia resulting from the challenge virus. This statement is supported by the fact that when the vaccinal strain was inoculated into pigs, and not inactivated, no cPCV1-2 viremia was detected using specific primers (Fenaux, M., Opriessnig, T., Halbur, P. G., Elvinger, F., Meng, X. J. A chimeric porcine circovirus (PCV) with the immunogenic gene of the pathogenic PCV type 2 (PCV2) cloned into the genomic backbone of the non-pathogenic PCV1 induces protective immunity against PCV2 infection in pigs. J. Virol.; 78(12): 6297-6303, 2004).

The amount of PCV2 genome detected in serum was drastically reduced in vaccinated, revaccinated and challenged pigs (group 2,2-shot) and prevented in vaccinated and challenged pigs (group 1,1-shot). In contrast, non-vaccinated and challenged pigs (group 3) presented high amounts of PCV2 genome copies per ml of serum. No viremic pigs were detected during the complete PI period in pigs vaccinated 1-shot and challenged, and only 1 pig was viremic (D14 PI) in pigs vaccinated 2-shot and challenged. These results are equivalent to those obtained using the cPCV1-2 virus as a live vaccine (Fenaux, M., Opriessnig, T., Halbur, P. G., Elvinger, F., Meng, X. J. A chimeric porcine circovirus (PCV) with the immunogenic gene of the pathogenic PCV type 2 (PCV2) cloned into the genomic backbone of the non-pathogenic PCV1 induces protective immunity against PCV2 infection in pigs. J. Virol.; 78(12): 6297-6303, 2004).

PCV2 nucleic acid was detected in tissues of 33.3% (7 out of 21) of the non-vaccinated and challenged pigs (score 1). In contrast, none of vaccinated and challenged pigs had PCV2 nucleic acid within tissues. These results are in accordance to those obtained with the cPCV1-2 virus used as a live vaccine (Fenaux, M., Opriessnig, T., Halbur, P. G., Elvinger, F., Meng, X. J. A chimeric porcine circovirus (PCV) with the immunogenic gene of the pathogenic PCV type 2 (PCV2) cloned into the genomic backbone of the non-pathogenic PCV1 induces protective immunity against PCV2 infection in pigs. J. Virol.; 78(12): 6297-6303, 2004).

Gross lesions did not allow for the evaluation of the vaccine, since very few pigs presented gross lesions in all groups examined, and those lesions observed could be attributed to other pathologies, in certain cases. Consequently, no differences were detected between groups.

With respect to microscopic lesions, there was a reduction in the mean score obtained for vaccinated and challenged pigs (0.06 in group 1, and 0.07 in group 2), compared to non-vaccinated and challenged pigs (0.38). In this latter group, there were 8 pigs with a score of 1 (38.09%). In contrast, in vaccinated and challenged pigs, there was only 1 pig in each group with a score of 1 (5.88 and 7.14%, respectively).

Since only lesions of score 1, which are typical of subclinical PCV2 infections (Krakowka, S., Ellis, J., McNeilly, F., Waldner, C., Allan, G. Features of porcine circovirus-2 disease: correlations between lesions, amount and distribution of virus, and clinical outcome. J. Vet. Diagn. Invest.; 17: 213-222, 2005) were developed in non-vaccinated and challenged pigs, it was not possible to know the effect of the vaccine in preventing the development of lesions of preclinical PMWS (score 2). Based on the present results, it can be said that vaccination 4 months prior to challenge is able to prevent the development of lesions associated with subclinical cases of PMWS.

### SUMMARY

The vaccine cPCV1-2 (KV), when administered in 1-shot to 3-4 week-old pigs, or as 2-shots at 3-4 weeks and 6-7 weeks of age, is able to prevent viremia associated with PCV2 infection. Statistically significant differences were detected between groups 1, 2 and 3, at days 7, 14 and 21 PI.

At necropsy, gross lesions did not allow for evaluation of the vaccine, since very few pigs presented gross lesions in all groups examined, and those lesions observed could be, in some cases, attributed to other pathologies.

However, at the microscopic level, the development of lesions (mainly in lymphoid tissues) typical of PCV2 infection were reduced in vaccinated animals: in the non-vaccinated and challenged group, 38.09% of the pigs presented mild lymphocyte depletion and infiltration, while in the vaccinated and challenged groups (1-shot and 2-shots), this was only observed in one pig from each group (5.88 and 7.14%, respectively).

The presence of the PCV2 genome in target tissues was detected by ISH in 33.3% of the non-vaccinated and challenged pigs. In contrast, none of vaccinated and challenged pigs had PCV2 nucleic acid within tissues

The chimeric porcine circovirus type 1-type 2 (cPCV1-2) killed and adjuvanted vaccine is effective in protecting pigs against the adverse effects of PCV2 infection (PCV2 viremia, lymphoid tissue lesions and presence of the PCV2 genome in tissues), even when administered 4 months prior to challenge.

The vaccine is also able to provide cross-protection against the high virulence/high mortality type 2B European strains of porcine circovirus.

What is claimed is:

1. A method of immunizing a pig against viral infection or postweaning multisystemic wasting syndrome (PMWS) caused by a high virulence/high mortality strain of PCV2 comprising administering to the pig an immunogenically effective amount of a vaccine composition comprising:
(a) an immunogenically effective amount of a type 1-type 2 chimeric porcine circovirus (PCV1-2) comprising a nucleic acid molecule encoding an infectious, nonpathogenic PCV1 which contains an immunogenic open reading frame (ORF) gene of a pathogenic PCV2 in place of an ORF gene of the PCV1 nucleic acid molecule; or
(b) a nucleic acid molecule encoding the type 1-type 2 chimeric porcine circovirus of a).

2. The method of claim 1, wherein the vaccine further comprises an adjuvant.

3. The method of claim 1, wherein the ORF gene is ORF-2.

4. The method of claim 3, wherein the ORF-2 gene from the PCV-2 strain comprises the nucleotide sequence as set forth in SEQ ID NO: 3.

5. The method of claim 4, wherein the protein encoded by the ORF-2 gene comprises the amino acid sequence as set forth in SEQ ID NO: 4.

6. The method of claim 1, wherein the vaccine comprises the nucleotide sequence as set forth in SEQ ID NO: 1, its complementary strand, or a nucleic acid sequence having at least 95% homology to the nucleotide sequence of SEQ ID NO: 1.

7. The method of claim 1, wherein the vaccine comprises a killed/inactivated, or a live-attenuated, chimeric porcine circovirus and a non-toxic, physiologically acceptable carrier.

8. The method of claim 1, wherein the vaccine is administered parenterally.

9. The method of claim 8, wherein the vaccine is administered subcutaneously, intramuscularly, intranasally, transdermally, intrahepatically, or via the intralymphoid route.

10. The method of claim 1, wherein the vaccine is administered as a single dose, or as multiple doses.

11. The method of claim 1, wherein the method results in induction of a humoral or a cell-mediated immune response.

12. The method of claim 11, wherein the immune response is observed for a period of at least four months.

13. A method for reducing the mortality in pigs associated with a high virulence/high mortality strain of a type 2B porcine circovirus comprising administering an immunogenically effective amount of a type 1-type 2 chimeric porcine circovirus vaccine composition to a pig, wherein the vaccine composition comprises:
(a) an immunogenically effective amount of a type 1-type 2 chimeric porcine circovirus (PCV1-2) comprising a nucleic acid molecule encoding an infectious, nonpathogenic PCV1 which contains an immunogenic open reading frame (ORF) gene of a pathogenic PCV2 in place of an ORF gene of the PCV1 nucleic acid molecule; or
(b) a nucleic acid molecule encoding the type 1-type 2 chimeric porcine circovirus of a).

14. The method of claim 13, wherein the immunogenic ORF gene is ORF-2.

15. The method of claim 14, wherein the ORF-2 gene from the PCV-2 strain comprises the nucleotide sequence as set forth in SEQ ID NO: 3.

16. The method of claim 15, wherein the protein encoded by the ORF-2 gene from the PCV-2 strain comprises the amino acid sequence as set forth in SEQ ID NO: 4.

17. The method of claim 13, wherein the vaccine comprises the nucleotide sequence as set forth in SEQ ID NO: 1, its complementary strand, or a nucleic acid sequence having at least 95% homology to the nucleotide sequence of SEQ ID NO: 1.

18. The method of claim 13, wherein the vaccine comprises a killed/inactivated, or a live-attenuated, chimeric porcine circovirus and a non-toxic, physiologically acceptable carrier.

19. The method of claim 13, wherein the vaccine is administered parenterally.

20. The method of claim 19, wherein the vaccine is administered subcutaneously, intramuscularly, intranasally, transdermally, intrahepatically, or via the intralymphoid route.

21. The method of claim 13, wherein the vaccine is administered in one dose or in multiple doses.

22. The method of claim 13, wherein said reducing the mortality in pigs is the result of generating a cross-protective humoral or a cell-mediated immune response.

23. The method of claim 22, wherein the cross-protective immune response is observed for a period of at least four months.

24. The method of claim 13, wherein the type-2B porcine circovirus shares at least 80% nucleic acid sequence homology with a type-2A strain of porcine circovirus.

25. The method of claim 24, wherein the type-2B porcine circovirus shares at least 95% nucleic acid sequence homology with a type-2A strain of porcine circovirus.

26. The method of claim 25, wherein the type-2B porcine circovirus shares at least 97% nucleic acid sequence homology with a type-2A strain of porcine circovirus.

27. The method of claim 26, wherein the type-2B porcine circovirus shares at least 99% nucleic acid sequence homology with a type-2A strain of porcine circovirus.

28. The method of any one of claims 24-27, wherein the type-2A porcine circovirus comprises the nucleotide sequence of any one or more of SEQ ID NOs: 5, 7 or 9.

29. The method of claim 13, wherein the type-2B porcine circovirus contains a capsid protein encoded by an ORF 2 gene, wherein the capsid protein exhibits not less than 90% sequence identity with a capsid protein encoded by an ORF 2 gene of a type 2A strain of a porcine circovirus.

30. The method of claim 13, wherein a type-2B porcine circovirus contains a capsid protein encoded by an ORF 2 gene, wherein the capsid protein exhibits not less than 90% sequence identity with the amino acid sequence of SEQ ID NO: 4.

31. The method of claim 29, wherein the ORF2 gene is from a type 2B strain of porcine circovirus, wherein the type 2B strain comprises the nucleic acid sequence of any one of SEQ ID NOs: 11, 13, 15 or 17 and wherein the ORF 2 gene is from a type 2A strain of a porcine circovirus, wherein the type 2A strain comprises the nucleic acid of any one of SEQ ID NOs: 5, 7 or 9.

32. The method of claim 29, wherein the capsid protein encoded by the ORF 2 gene from a type 2B strain of porcine circovirus comprises the amino acid sequence of any one of SEQ ID NOs: 12, 14, 16 or 18 and wherein the capsid protein encoded by the ORF 2 gene from a type 2A strain of a porcine circovirus comprises the amino acid sequence of any one of SEQ ID NOs: 6, 8 or 10.

33. The method of either of claims 1 or 13, wherein the administering of the vaccine results in amelioration of one or more of the following clinical symptoms:
(a) reduction of microscopic lesions in one or more tissues of pigs exposed to a virulent form of a type-2B porcine circovirus;
(b) reduction of viremia associated with a porcine circovirus infection;
(c) reduction in the level of type-2A or type-2B nucleic acid in one or more tissues.

34. The method of claim 33, wherein the tissues are lymphoid or non-lymphoid tissues.

35. The method of either one of claims 1 or 13, wherein the method further comprises administering an immunogenically effective amount of a second different vaccine prior to, in conjunction with, or subsequent to, administering the type-1-type 2 chimeric porcine circovirus vaccine composition.

36. The method of claim 35, wherein the second different vaccine is protective against a microorganism selected from the group consisting of porcine reproductive and respiratory syndrome virus (PRRS), porcine parvovirus (PPV), Mycoplasma hyopneumoniae, Haemophilus parasuis, Pasteurella multocida, Streptococcum suis, Actinobacillus pleuropneumoniae, Bordetella bronchiseptica, Salmonella choleraesuis, Erysipelothrix rhusiopathiae, leptospira bacteria, swine influenza virus, Escherichia coli antigen, porcine respiratory coronavirus, rotavirus, a pathogen causative of Aujesky's Disease, and a pathogen causative of Swine Transmissible Gastroenteritis.

37. The method of claim 29, wherein the capsid protein encoded by the ORF 2 gene of a type-2B porcine circovirus has a conservative or non-conservative amino acid substitution at one or more of the following positions of any one of SEQ ID NOs: 6, 8 or 10: position numbers 57, 59, 63, 75, 77, 80, 86, 88, 89, 91, 99, 121, 151, 190, 191, 200, 206, 210, 232.

38. The method of claim 29, wherein the capsid protein encoded by the ORF 2 gene of a type-2B porcine circovirus has one or more of the following variations:
(a) the isoleucine at position 91 of any one of SEQ ID NOs: 6, 8 or 10 is replaced with a valine; and/or
(b) the lysine at position 99 of SEQ ID NO: 6 is replaced with an arginine.

39. A method of immunizing a pig against viral infection or postweaning multisystemic wasting syndrome (PMWS) caused by a high virulence strain of a type 2 porcine circovirus (PCV2) comprising administering to the pig an immunogenically effective amount of an immunogenic composition comprising an ORF2 polypeptide from a type 2A porcine circovirus, or a nucleic acid encoding the ORF2 polypeptide from a type 2A porcine circovirus, and a pharmaceutically acceptable carrier, wherein the administering of the composition to a pig induces a cross-protective immune response against a high virulence strain of a type 2 porcine circovirus.

40. The method of claim 39, wherein the high virulence strain of a type 2 porcine circovirus is a type 2B strain.

41. The method of claim 39, wherein the immunogenic composition further comprises an adjuvant.

42. The method of claim 39, wherein the ORF2 polypeptide from the type 2A porcine circovirus comprises the amino acid sequence of any one of SEQ ID NOs: 4, 6, 8 or 10.

43. The method of claim 39, wherein the ORF2 polypeptide from the type 2A porcine circovirus has at least 90% sequence identity to the amino acid sequence of any one of SEQ ID NOs: 4, 6, 8 or 10.

44. An immunogenic composition comprising an immunogenically effective amount of an ORF2 polypeptide from a type 2A porcine circovirus, or a nucleic acid encoding the ORF2 polypeptide from a type 2A porcine circovirus, and a pharmaceutically acceptable carrier, wherein the administering of the composition to a pig induces a cross-protective immune response against a high virulence strain of a type 2B porcine circovirus.
